SEQUENCE LISTING (46 sequences)

SEQ ID NO: 1 - protein, 245 aa, synthetic construct
VKLVESGGGLVKPGGSLKLSCAASGFTFSTYAMSWVRQTPEKRLEWVASISSGGNTYYPDSVKGRFTISRDNARHILYLQMSSLRSEDTAMYYCARDSYYFGNSVYYAMDYWGQGTSVTVSSGGGGSGGGGSGGGGSDIKMTQSPSSMYASLGERVTITCKASQDINSYFSWFQQKPGKSPKTLIYRANRLVDGVPSRFSGSGSGQDYSLTISSLEYEDMGIYYCLQYDEFPYTFGGGTKLEIKR

SEQ ID NO: 2 - protein, 122 aa, synthetic construct
VKLVESGGGLVKPGGSLKLSCAASGFTFSTYAMSWVRQTPEKRLEWVASISSGGNTYYPDSVKGRFTISRDNARHILYLQMSSLRSEDTAMYYCARDSYYFGNSVYYAMDYWGQGTSVTVSS

SEQ ID NO: 3 - protein, 108 aa, synthetic construct
DIKMTQSPSSMYASLGERVTITCKASQDINSYFSWFQQKPGKSPKTLIYRANRLVDGVPSRFSGSGSGQDYSLTISSLEYEDMGIYYCLQYDEFPYTFGGGTKLEIKR

SEQ ID NO: 4 - protein, 500 aa, synthetic construct
MALPVTALLLPLALLLHAARPASVKLVESGGGLVKPGGSLKLSCAASGFTFSTYAMSWVRQTPEKRLEWVASISSGGNTYYPDSVKGRFTISRDNARHILYLQMSSLRSEDTAMYYCARDSYYFGNSVYYAMDYWGQGTSVTVSSGGGGSGGGGSGGGGSDIKMTQSPSSMYASLGERVTITCKASQDINSYFSWFQQKPGKSPKTLIYRANRLVDGVPSRFSGSGSGQDYSLTISSLEYEDMGIYYCLQYDEFPYTFGGGTKLEIKRLEKPTTTPAPRPPTPAPTIASQPLSLRPEASRPAAGGAVHTRGLDFASDKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 5 - protein, 123 aa, synthetic construct
EVKLVESGGGLVKPGGSLKLSCAASGFTFSTYAMSWVRQTPEKRLEWVASISSGGNTYYPDSVKGRFTISRDNARHILYLQMSSLRSEDTAMYYCARDSYYFGNSVYYAMDYWGQGTSVTVSS

SEQ ID NO: 6 - protein, 107 aa, synthetic construct
DIKMTQSPSSMYASLGERVTITCKASQDINSYFSWFQQKPGKSPKTLIYRANRLVDGVPSRFSGSGSGQDYSLTISSLEYEDMGIYYCLQYDEFPYTFGGGTKLEIK

SEQ ID NO: 7 - protein, 501 aa, synthetic construct
MALPVTALLLPLALLLHAARPASEVKLVESGGGLVKPGGSLKLSCAASGFTFSTYAMSWVRQTPEKRLEWVASISSGGNTYYPDSVKGRFTISRDNARHILYLQMSSLRSEDTAMYYCARDSYYFGNSVYYAMDYWGQGTSVTVSSGGGGSGGGGSGGGGSDIKMTQSPSSMYASLGERVTITCKASQDINSYFSWFQQKPGKSPKTLIYRANRLVDGVPSRFSGSGSGQDYSLTISSLEYEDMGIYYCLQYDEFPYTFGGGTKLEIKLEKPTTTPAPRPPTPAPTIASQPLSLRPEASRPAAGGAVHTRGLDFASDKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 8 - protein, 245 aa, synthetic construct
EVQLVESGGGLIQPGGSLRLSCAASGFTFSTYAMSWVRQAPGKGLEWVSSISSGGNTYYADSVKGRFTISRDNSKNTLYLQMNSLRAEDTAVYYCARDSYYFGNSVYYAMDYWGAGTTVTVSSGGGGSGGGGSGGGGSDIQMTQSPSSLSASVGDRVTITCKASQDINSYFSWFQQKPGKAPKSLIYRANRLVSGVPSRFSGSGSGTDFTLTISSLQPEDFATYYCLQYDEFPYTFGGGTRLEIK

SEQ ID NO: 9 - protein, 121 aa, synthetic construct
EVQLVESGGGLIQPGGSLRLSCAASGFTFSTYAMSWVRQAPGKGLEWVSSISSGGNTYYADSVKGRFTISRDNSKNTLYLQMNSLRAEDTAVYYCARDSYYFGNSVYYAMDYWGAGTTVTV

SEQ ID NO: 10 - protein, 107 aa, synthetic construct
DIQMTQSPSSLSASVGDRVTITCKASQDINSYFSWFQQKPGKAPKSLIYRANRLVSGVPSRFSGSGSGTDFTLTISSLQPEDFATYYCLQYDEFPYTFGGGTRLEIK

SEQ ID NO: 11 - protein, 501 aa, synthetic construct
MALPVTALLLPLALLLHAARPASEVQLVESGGGLIQPGGSLRLSCAASGFTFSTYAMSWVRQAPGKGLEWVSSISSGGNTYYADSVKGRFTISRDNSKNTLYLQMNSLRAEDTAVYYCARDSYYFGNSVYYAMDYWGAGTTVTVSSGGGGSGGGGSGGGGSDIQMTQSPSSLSASVGDRVTITCKASQDINSYFSWFQQKPGKAPKSLIYRANRLVSGVPSRFSGSGSGTDFTLTISSLQPEDFATYYCLQYDEFPYTFGGGTRLEIKLEKPTTTPAPRPPTPAPTIASQPLSLRPEASRPAAGGAVHTRGLDFASDKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 12 - protein, 245 aa, synthetic construct
QVQLVESGGGVVQPGRSLRLSCAASGFTFSTYAMSWVRQAPGKGLEWVASISSGGNTYYPDSVKGRFTISRDNSKNTLYLQMNSLRAEDTAVYYCARDSYYFGNSVYYAMDYWGAGTTVTVSSGGGGSGGGGSGGGGSDIQMTQSPSSLSASVGDRVTITCKASQDINSYFSWFQQKPGKAPKSLIYRANRLVSGVPSRFSGSGSGTDFTLTISSLQPEDFATYYCLQYDEFPYTFGGGTRLEIK

SEQ ID NO: 13 - protein, 123 aa, synthetic construct
QVQLVESGGGVVQPGRSLRLSCAASGFTFSTYAMSWVRQAPGKGLEWVASISSGGNTYYPDSVKGRFTISRDNSKNTLYLQMNSLRAEDTAVYYCARDSYYFGNSVYYAMDYWGAGTTVTVSS

SEQ ID NO: 14 - protein, 107 aa, synthetic construct; identical to SEQ ID NO: 10

SEQ ID NO: 15 - protein, 501 aa, synthetic construct
MALPVTALLLPLALLLHAARPASQVQLVESGGGVVQPGRSLRLSCAASGFTFSTYAMSWVRQAPGKGLEWVASISSGGNTYYPDSVKGRFTISRDNSKNTLYLQMNSLRAEDTAVYYCARDSYYFGNSVYYAMDYWGAGTTVTVSSGGGGSGGGGSGGGGSDIQMTQSPSSLSASVGDRVTITCKASQDINSYFSWFQQKPGKAPKSLIYRANRLVSGVPSRFSGSGSGTDFTLTISSLQPEDFATYYCLQYDEFPYTFGGGTRLEIKLEKPTTTPAPRPPTPAPTIASQPLSLRPEASRPAAGGAVHTRGLDFASDKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 16 - protein, 245 aa, synthetic construct
QVQLVESGGGVVQPGRSLRLSCAASGFTFSTYAMSWVRQAPGKGLEWVASISSGGNTYYPDSVKGRFTISRDNSKNTLYLQMNSLRAEDTAVYYCARDSYYFGNSVYYAMDYWGAGTTVTVSSGGGGSGGGGSGGGGSDIQMTQSPSSLSASVGDRVTITCKASQDINSYFSWYQQKPGKAPKLLIYRANRLVTGVPSRFSGSGSGTDFTFTISSLQPEDIATYYCLQYDEFPYTFGGGTRLEIK

SEQ ID NO: 17 - protein, 123 aa, synthetic construct; identical to SEQ ID NO: 13

SEQ ID NO: 18 - protein, 107 aa, synthetic construct
DIQMTQSPSSLSASVGDRVTITCKASQDINSYFSWYQQKPGKAPKLLIYRANRLVTGVPSRFSGSGSGTDFTFTISSLQPEDIATYYCLQYDEFPYTFGGGTRLEIK

SEQ ID NO: 19 - protein, 501 aa, synthetic construct
MALPVTALLLPLALLLHAARPASQVQLVESGGGVVQPGRSLRLSCAASGFTFSTYAMSWVRQAPGKGLEWVASISSGGNTYYPDSVKGRFTISRDNSKNTLYLQMNSLRAEDTAVYYCARDSYYFGNSVYYAMDYWGAGTTVTVSSGGGGSGGGGSGGGGSDIQMTQSPSSLSASVGDRVTITCKASQDINSYFSWYQQKPGKAPKLLIYRANRLVTGVPSRFSGSGSGTDFTFTISSLQPEDIATYYCLQYDEFPYTFGGGTRLEIKLEKPTTTPAPRPPTPAPTIASQPLSLRPEASRPAAGGAVHTRGLDFASDKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 20 - DNA, 735 bp, synthetic construct
  source 1..735: mol_type = other DNA
gtgaagctggtggagtctgggggaggcttagtgaagcctggagggtccctgaaactctcctgtgcagcctctggattcactttcagtacctatgccatgtcttgggttcgccagactccagagaagaggctggagtgggtcgcatccattagtagtggtggtaacacctactatccagacagtgtgaagggccgattcaccatctccagagataatgccaggcacatcctgtacctgcaaatgagcagtctgaggtctgaggacacggccatgtattactgtgcaagagattcttattacttcggtaatagcgtttactatgctatggactactggggtcaaggaacctcagtcaccgtctcctcaggtggcggtggttctggtggcggtggttctggtggcggtggttctgacatcaagatgacccagtctccatcttccatgtatgcatctctaggagagagagtcactatcacttgcaaggcgagtcaggacattaatagctattttagctggttccagcaaaaaccagggaaatctcctaagaccctgatctatcgtgcaaatagattggtagatggggtcccatcaaggttcagtggcagtggatctgggcaggattattctctcaccatcagcagcctggagtatgaagatatgggaatttattattgtctacagtatgatgagtttccgtacacgttcggaggggggaccaaactggaaataaaacgg

SEQ ID NO: 21 - DNA, 63 bp, synthetic construct
  source 1..63: mol_type = other DNA
atggccttaccagtgaccgccttgctcctgccgctggccttgctgctccacgccgccaggccg

SEQ ID NO: 22 - protein, 21 aa, synthetic construct
MALPVTALLLPLALLLHAARP

SEQ ID NO: 23 - protein, 245 aa, synthetic construct
EVKLVESGGGLVKPGGSLKLSCAASGFTFSTYAMSWVRQTPEKRLEWVASISSGGNTYYPDSVKGRFTISRDNARHILYLQMSSLRSEDTAMYYCARDSYYFGNSVYYAMDYWGQGTSVTVSSGGGGSGGGGSGGGGSDIKMTQSPSSMYASLGERVTITCKASQDINSYFSWFQQKPGKSPKTLIYRANRLVDGVPSRFSGSGSGQDYSLTISSLEYEDMGIYYCLQYDEFPYTFGGGTKLEIK

SEQ ID NO: 24 - DNA, 141 bp, synthetic construct
  source 1..141: mol_type = other DNA
aagcccaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgat

SEQ ID NO: 25 - protein, 47 aa, synthetic construct
KPTTTPAPRPPTPAPTIASQPLSLRPEASRPAAGGAVHTRGLDFASD

SEQ ID NO: 26 - DNA, 204 bp, synthetic construct
  source 1..204: mol_type = other DNA
ttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 27 - protein, 68 aa, synthetic construct
FWVLVVVGGVLACYSLLVTVAFIIFWVRSKRSRLLHSDYMNMTPRRPGPTRKHYQPYAPPRDFAAYRS

SEQ ID NO: 28 - DNA, 339 bp, synthetic construct
  source 1..339: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 29 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 30 - DNA, 1503 bp, synthetic construct
  source 1..1503: mol_type = other DNA
atggccttaccagtgaccgccttgctcctgccgctggccttgctgctccacgccgccaggccggctagcgtgaagctggtggagtctgggggaggcttagtgaagcctggagggtccctgaaactctcctgtgcagcctctggattcactttcagtacctatgccatgtcttgggttcgccagactccagagaagaggctggagtgggtcgcatccattagtagtggtggtaacacctactatccagacagtgtgaagggccgattcaccatctccagagataatgccaggcacatcctgtacctgcaaatgagcagtctgaggtctgaggacacggccatgtattactgtgcaagagattcttattacttcggtaatagcgtttactatgctatggactactggggtcaaggaacctcagtcaccgtctcctcaggtggcggtggttctggtggcggtggttctggtggcggtggttctgacatcaagatgacccagtctccatcttccatgtatgcatctctaggagagagagtcactatcacttgcaaggcgagtcaggacattaatagctattttagctggttccagcaaaaaccagggaaatctcctaagaccctgatctatcgtgcaaatagattggtagatggggtcccatcaaggttcagtggcagtggatctgggcaggattattctctcaccatcagcagcctggagtatgaagatatgggaatttattattgtctacagtatgatgagtttccgtacacgttcggaggggggaccaaactggaaataaaacggctcgagaagcccaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgataagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 31 - DNA, 1505 bp, synthetic construct
  source 1..1505: mol_type = other DNA
atggccttaccagtgaccgccttgctcctgccgctggccttgctgctccacgccgccaggccggctagcgaagtgaagcttgtcgaatccggcggtggattggttaaaccaggcggaagtttgaaactgagttgtgctgcttctggttttacctttagcacatacgctatgtcctgggttaggcagacgccggagaaacgattggagtggtagcatctatttcttctggcggcaatacttattaccctgacagcgtgaaaggccggttcactatttctcgagataatgcgcggcacatactctatctccagatgtcttctctccgctcagaggatacagcgatgtactattgtgcaagggatagttactatttcggaaactctgtgtattatgcaatggattactggggtcagggaacttcagtcacagtaagctcaggtgggggaggaagcggcggtggcggctcagggggaggtggatctgatattaaaatgactcagtctccatcaagcatgtacgcctctctgggagagcgagttactattacctgtaaagcatcacaagatattaactcttattttagttggtttcaacaaaagcctggaaaatcacctaaaactttgatttatagagccaataggcttgtggatggtgtacctagtcggtttagcggctcagggtcaggccaagactattctttgaccatctcttctctggagtatgaggacatgggaatctattactgtcttcagtacgatgagttcccctatacgtttggtggaggcactaaattggagattaaactcgagaagcccaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgataagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctctacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 32 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 33 - protein, 42 aa, synthetic construct
KRGRKKLLYIFKQPFMRPVQTTQEEDGCSCRFPEEEEGGCEL

SEQ ID NO: 34 - DNA, 735 bp, synthetic construct
  source 1..735: mol_type = other DNA
gaagtacagcttgttgaatcaggtggtggtcttattcagccaggaggctccttgcgactgagctgtgccgcttctgggttcacctttagcacttacgcaatgagttgggtccgacaagccccaggtaagggattggaatgggtaagttccatttccagcggagggaacacttattacgccgattctgtgaaaggacgctttactatatcccgagacaatagtaaaaacacattgtatttgcaaatgaactctttgagggccgaggacactgccgtctactattgtgcccgcgacagctattatttcggcaactctgtgtattacgcgatggattactggggtgccggcacaactgtcaccgtttcatctggcggaggaggcagtggcggagggggctcaggcggtggtggaagtgatattcaaatgacccaatcaccctcttcattgtctgcaagcgtaggtgaccgagtcacgataacctgcaaagcctctcaagatattaattcatacttttcttggtttcaacaaaaaccgggaaaggcgcctaagtcattgatttaccgcgcgaaccggttggtatcaggagtaccgtcaagattctcagggagtgggtcaggcacagatttcacactcactatttcttccttgcaacctgaagacttcgcaacctattattgcttgcagtatgatgagtttccgtacactttcggggggggtacaaggctggagatcaaa

SEQ ID NO: 35 - DNA, 1506 bp, synthetic construct
  source 1..1506: mol_type = other DNA
atggccttaccagtgaccgccttgctcctgccgctggccttgctgctccacgccgccaggccggctagcgaagtacagcttgttgaatcaggtggtggtcttattcagccaggaggctccttgcgactgagctgtgccgcttctgggttcacctttagcacttacgcaatgagttgggtccgacaagccccaggtaagggattggaatgggtaagttccatttccagcggagggaacacttattacgccgattctgtgaaaggacgctttactatatcccgagacaatagtaaaaacacattgtatttgcaaatgaactctttgagggccgaggacactgccgtctactattgtgcccgcgacagctattatttcggcaactctgtgtattacgcgatggattactggggtgccggcacaactgtcaccgtttcatctggcggaggaggcagtggcggagggggctcaggcggtggtggaagtgatattcaaatgacccaatcaccctcttcattgtctgcaagcgtaggtgaccgagtcacgataacctgcaaagcctctcaagatattaattcatacttttcttggtttcaacaaaaaccgggaaaggcgcctaagtcattgatttaccgcgcgaaccggttggtatcaggagtaccgtcaagattctcagggagtgggtcaggcacagatttcacactcactatttcttccttgcaacctgaagacttcgcaacctattattgcttgcagtatgatgagtttccgtacactttcggggggggtacaaggctggagatcaaactcgagaagcccaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgataagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctctacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 36 - DNA, 735 bp, synthetic construct
  source 1..735: mol_type = other DNA
caggtacaattggtagagtccggcggaggggttgttcagccaggacggtccttgcggttgtcttgtgctgcgtcaggattcacattctcaacgtacgcgatgtcttgggtgcgccaagctcccggtaaagggctggaatgggtggcctcaatctcatctggagggaacacttactaccctgatagtgttaaaggtcgctttactatctcaagggacaatagcaagaataccttgtatctgcaaatgaactcacttagagcagaggacacagcggtatattactgtgctagagactcatattatttcggcaactccgtttattacgcgatggattactggggcgcagggactacggtaactgtatcttctggtggtggagggtctgggggcgggggtagtggcggcggtggcagtgacatccagatgacacagtctccgtcttcattgagtgcaagcgtcggcgatcgggttaccattacgtgtaaggcaagtcaggacatcaacagttatttttcatggtttcaacaaaagcctggaaaagcgccgaaatcactcatttaccgagctaataggcttgtctctggcgttccgtctcgcttcagtggaagtgggagcggtactgattttaccctcaccatatcaagccttcaaccggaggattttgccacgtactattgtctccagtacgatgaatttccatatacgtttggcggcgggactcgcttggagattaaa

SEQ ID NO: 37 - DNA, 1506 bp, synthetic construct
  source 1..1506: mol_type = other DNA
atggccttaccagtgaccgccttgctcctgccgctggccttgctgctccacgccgccaggccggctagccaggtacaattggtagagtccggcggaggggttgttcagccaggacggtccttgcggttgtcttgtgctgcgtcaggattcacattctcaacgtacgcgatgtcttgggtgcgccaagctcccggtaaagggctggaatgggtggcctcaatctcatctggagggaacacttactaccctgatagtgttaaaggtcgctttactatctcaagggacaatagcaagaataccttgtatctgcaaatgaactcacttagagcagaggacacagcggtatattactgtgctagagactcatattatttcggcaactccgtttattacgcgatggattactggggcgcagggactacggtaactgtatcttctggtggtggagggtctgggggcgggggtagtggcggcggtggcagtgacatccagatgacacagtctccgtcttcattgagtgcaagcgtcggcgatcgggttaccattacgtgtaaggcaagtcaggacatcaacagttatttttcatggtttcaacaaaagcctggaaaagcgccgaaatcactcatttaccgagctaataggcttgtctctggcgttccgtctcgcttcagtggaagtgggagcggtactgattttaccctcaccatatcaagccttcaaccggaggattttgccacgtactattgtctccagtacgatgaatttccatatacgtttggcggcgggactcgcttggagattaaactcgagaagcccaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgataagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctctacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 38 - DNA, 735 bp, synthetic construct
  source 1..735: mol_type = other DNA
caggtacaactggtggaatccggcgggggagtagtacagcccggacgatctcttcgactctcatgtgcagcgtccgggttcactttttctacctacgcaatgtcatgggtacgacaggcgccgggcaaaggcctcgaatgggttgcatccatttcatcaggaggtaatacatattatcctgattcagtcaagggccgattcacgattagtcgagataatagcaagaacactctctacttgcagatgaactccctgcgggctgaggacacggccgtgtattattgcgctcgcgatagttattacttcggcaattccgtatattatgcgatggactattggggcgccggtactaccgtgactgtttcctctggtgggggtgggtccgggggcggtggttcaggtggaggcggatccgacattcaaatgacccagtctccctcaagtttgtctgcatctgttggcgatagagttacaataacatgcaaagccagtcaagacatcaactcatacttctcctggtatcaacaaaagccaggaaaagctccgaaactgttgatctaccgggccaaccggctggtcactggcgtgccatcccggttcagtggcagcggaagcggaacagatttcacgtttaccatctctagcctccaaccggaggacatcgcaacatactattgccttcagtatgatgagtttccctacactttcggtggcggcacccgacttgagatcaaa

SEQ ID NO: 39 - DNA, 1506 bp, synthetic construct
  source 1..1506: mol_type = other DNA
atggccttaccagtgaccgccttgctcctgccgctggccttgctgctccacgccgccaggccggctagccaggtacaactggtggaatccggcgggggagtagtacagcccggacgatctcttcgactctcatgtgcagcgtccgggttcactttttctacctacgcaatgtcatgggtacgacaggcgccgggcaaaggcctcgaatgggttgcatccatttcatcaggaggtaatacatattatcctgattcagtcaagggccgattcacgattagtcgagataatagcaagaacactctctacttgcagatgaactccctgcgggctgaggacacggccgtgtattattgcgctcgcgatagttattacttcggcaattccgtatattatgcgatggactattggggcgccggtactaccgtgactgtttcctctggtgggggtgggtccgggggcggtggttcaggtggaggcggatccgacattcaaatgacccagtctccctcaagtttgtctgcatctgttggcgatagagttacaataacatgcaaagccagtcaagacatcaactcatacttctcctggtatcaacaaaagccaggaaaagctccgaaactgttgatctaccgggccaaccggctggtcactggcgtgccatcccggttcagtggcagcggaagcggaacagatttcacgtttaccatctctagcctccaaccggaggacatcgcaacatactattgccttcagtatgatgagtttccctacactttcggtggcggcacccgacttgagatcaaactcgagaagcccaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgataagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctctacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 40 - skipped

SEQ ID NO: 41 - protein, 6 aa, synthetic construct
SSGGNT

SEQ ID NO: 42 - protein, 15 aa, synthetic construct
DSYYFGNSVYYAMDY

SEQ ID NO: 43 - protein, 6 aa, synthetic construct
QDINSY

SEQ ID NO: 44 - skipped

SEQ ID NO: 45 - protein, 9 aa, synthetic construct
LQYDEFPYT

SEQ ID NO: 46 - protein, 15 aa, synthetic construct
GGGGSGGGGSGGGGS

(19) United States
(12) Patent Application Publication
(10) Pub. No.: US 2025/0339465 A1
(43) Pub. Date: Nov. 6, 2025

(54) ANTI-ROR1 ANTIBODY AND ROR1-TARGETING ENGINEERED CELLS

(71) Applicant: Caribou Biosciences, Inc., Berkeley, CA (US)

(72) Inventors: Vita Golubovskaya, Pinole, CA (US); Lijun Wu, Albany, CA (US)

(73) Assignee: Caribou Biosciences, Inc., Berkeley, CA (US)

(21) Appl. No.: 18/868,087

(22) PCT Filed: May 22, 2023
(86) PCT No.: PCT/US2023/067314

§ 371 Date: Nov. 21, 2024

Related U.S. Application Data
(60) Provisional application No. 63/365,230, filed on May 24, 2022.

(51) Int. Cl. A61K 35/17; A61K 40/11; A61K 40/31; A61K 40/42; C07K 16/28; C12N 5/0783; C12N 15/86

(52) U.S. Cl. CPC A61K 35/17; A61K 40/11; A61K 40/31; A61K 40/421; C07K 16/2803; C12N 5/0636; C12N 15/86; A61K 2239/13; C07K 2317/24; C07K 2317/565; C07K 2317/622; C12N 2740/15043; C12N 2750/14143; C12N 2800/22

(57) ABSTRACT

The present invention is directed to a monoclonal mouse or humanized ROR1 antibody, or a single-chain variable fragment (scFv). The present invention is also directed to a mouse or humanized ROR1 chimeric antigen receptor (CAR) comprising from N-terminus to C-terminus: (i) a single-chain variable fragment (scFv) of the present invention, (ii) a transmembrane domain, (iii) at least one co-stimulatory domains, and (iv) an activating domain.

### CROSS-REFERENCE TO RELATED APPLICATIONS

This application claims priority to the U.S. Provisional application Ser. No. 63/365,230 filed on May 24, 2022, incorporated herein by reference.

### STATEMENT REGARDING FEDERALLY SPONSORED RESEARCH

None.

### SEQUENCE LISTING

The instant application contains a sequence listing which has been submitted electronically in XML format and is hereby incorporated by reference in its entirety. Said XML copy, created on May 22, 2023, is named CBI047.30_SL.xml and is 55,553 bytes in size.

### FIELD OF THE INVENTION

The present invention relates to the field of immunology and more specifically, to antibodies, T-cell receptors, and immune cells targeting ROR1, which are useful in the field of adoptive cellular immunotherapy for tumors.

### BACKGROUND OF THE INVENTION

Immunotherapy is emerging as a highly promising approach for the treatment of cancer. T cells or T lymphocytes, the armed forces of our immune system, constantly look for foreign antigens and discriminate abnormal (cancer or infected cells) from normal cells. Genetically modifying T cells or natural killer (NK) cells with CAR (chimeric antigen receptor) constructs is the most common approach to design tumor-specific T cells and NK cells. CAR-T cells and CAR-NK cells targeting tumor-associated antigens (TAA) can be infused into patients (called adoptive cell transfer or ACT) representing an efficient immunotherapy approach, see Grupp, et al., (2013) Chimeric antigen receptor-modified T cells for acute lymphoid leukemia. N Engl J Med 368, 1509-1518, and Maus, et al., (2013). T cells expressing chimeric antigen receptors can cause anaphylaxis in humans. Cancer Immunol Res 1, 26-31. The advantage of CAR-T (and CAR-NK) technology compared with chemotherapy or antibody is that engineered cells can proliferate and persist in the patient as a “living drug.” Maus, et al., (2014). Antibody-modified T cells: CARs take the front seat for hematologic malignancies. Blood 123, 2625-2635, and Goluboskaya et al., (2016) Different Subsets of T Cells, Memory, Effector Functions, and CAR-T Immunotherapy. Cancers (Basel). 2016 Mar. 15; 8 (3). pii: E36.

CARs usually consist of, in the N-C orientation, a monoclonal antibody-derived single-chain variable fragment (scFv), a hinge, a transmembrane domain, and one or more intracellular co-activation domains: e.g., CD8, CD28, CD137 (4-1BB), CD27; and one or more activation domains, e.g., CD3-zeta domain, see FIG. 1 and Maus (2013) and Maus (2014) supra. The evolution of CARs went from first generation (with no costimulatory domains) to second generation (with one co-stimulation domain) to third generation CAR (with several costimulatory domains). CAR-T cells with 3^{rd} generation CARs having multiple costimulatory domains possess increased cytolytic activity, and improved persistence resulting in augmented antitumor activity.

Natural killer cells (NK) cells are a type of cytotoxic lymphocyte critical to the innate immune system. The role NK cells play is analogous to that of cytotoxic T cells in the vertebrate adaptive immune response. NK cells provide rapid responses to virus-infected cells, acting at around 3 days after infection, and respond to tumor formation.

Tyrosine-protein kinase transmembrane receptor ROR1, also known as neurotrophic tyrosine kinase, receptor-related 1 (NTRKR1), is an enzyme that in humans is encoded by the ROR1 gene. ROR1 is a member of the receptor tyrosine kinase-like orphan receptor (ROR) family. ROR1 is 937 amino-acid protein, with amino acids 30-406 comprising the extracellular domain. The ROR1 gene encodes a receptor tyrosine kinase-like orphan receptor that modulates neurite growth in the central nervous system. ROR1 is a glycosylated type I membrane protein that belongs to the ROR subfamily of cell surface receptors. ROR1 is the receptor for ligand WNT5A which activates downstream NF kappa B signaling pathway and may result in the inhibition of WNT-mediated signaling. In addition, ROR1 has recently been shown to be expressed on ovarian cancer stem cells and promote migration, invasion and cancer stem cell spheroid formation. ROR1 is shown to be overexpressed in both hematological cancers and solid tumors that makes it a useful target for CAR-T therapy.

Low expression of ROR1 has been shown in most of normal human tissues such as adipose and soft tissue, bone marrow and immune system, endocrine tissues, female tissue, gastrointestinal tract, kidney and urinary bladder, liver and gallbladder, lung, muscle, male tissues, and skin.

### SUMMARY OF THE INVENTION

In some embodiments, the invention is anti-human ROR1 antibody or an antigen-binding fragment thereof comprising V_{H} having an amino acid sequence at least 90% identical to SEQ ID NO: 2 and V_{L} having an amino acid sequence at least 90% identical to SEQ ID NO: 3. In some embodiments, the anti-human ROR1 antibody or an antigen-binding fragment thereof comprises a humanized mouse amino acid sequence. In some embodiments, the antigen-binding fragment is a single-chain variable fragment (scFv). In some embodiments, the scFv comprises a V_{H} comprising SEQ ID NO: 17, a V_{L} comprising SEQ ID NO: 18, and a linker. In some embodiments, the scFv has a V_{H} consisting of SEQ ID NO: 17, a V_{L} consisting of SEQ ID NO: 18, and a linker. In some embodiments, the scFv is encoded by a nucleic acid comprising SEQ ID NO: 38. In some embodiments, the scFv comprises complementarity determining regions (CDRs) in the V_{H} and the V_{L}, wherein a CDR1 of the V_{H} comprises the sequence TYA, a CDR2 of the V_{H} comprises SEQ ID NO: 41, a CDR3 of the V_{H} comprises SEQ ID NO: 42, a CDR1 of the V_{L} comprises SEQ ID NO: 43, a CDR2 of the V_{L} comprises the sequence RAN, and a CDR3 of the V_{L} comprises SEQ ID NO: 45.

In some embodiments, the invention is a chimeric antigen receptor (CAR) comprising the scFv and further comprising: a transmembrane domain, at least one co-stimulatory domains, and an activation domain. In some embodiments, the co-stimulatory domain is CD28 or 4-1BB. In some embodiments, the activation domain is CD3 zeta. In some embodiments, the transmembrane domain is a CD8 transmembrane domain. In some embodiments, the CAR further comprises a signaling peptide and a hinge domain. In some embodiments, the signaling peptide and the hinge domain are the CD8 signaling peptide and the CD8 hinge domain. In some embodiments, the CAR comprises the amino acid sequence of SEQ ID NO: 19. In some embodiments, the CAR consists of the amino acid sequence of SEQ ID NO: 19. In some embodiments, the CAR is encoded by a nucleic acid comprising sequence of SEQ ID NO: 39.

In some embodiments, the invention is an engineered immune cell expressing the CAR of SEQ ID NO: 19. In some embodiments, the cell is selected from a CAR-T cell and a CAR-NK (natural killer) cell.
In some embodiments, the invention is a composition comprising the engineered immune expressing the CAR of SEQ ID NO: 19 and an excipient.

### BRIEF DESCRIPTION OF THE DRAWINGS

FIG. 1 is a diagram showing the first, second, and third generation chimeric antigen receptors (CARs) known in the art.

FIG. 2 is a diagram showing the structure of the anti-ROR1 CAR.

FIG. 3 is a Western blot demonstrating binding of the anti-ROR1 antibody to human ROR1 antigen.

FIG. 4 shows FACS data demonstrating staining of different cell lines with the anti-ROR1 antibody using different cancer cell lines.

FIG. 5 shows an RTCA assay demonstrating dose-dependent in cytotoxicity of anti-ROR1 CAR-T cells (ROR1-CD28-CD in panel A, and ROR1-4-1BB-CD3 in panel B) against the ROR1-expressing cell line SKOV-3.

FIG. 6 shows measurements of IFN-gamma secretion by anti-ROR1 CAR-T cells (ROR1-4-1BB-CD3) in the presence of SKOV-3 cells or control HL-60 cells (ROR1-negative cells).

FIG. 7. shows an RTCA assay demonstrating dose-dependent in vitro cytotoxicity of the anti-ROR1 CAR-T cells with humanized scFvs (PMC1182, PMC1183 and PMC1194 expressing PMC857, PMC858, and PMC862 anti-ROR1 CARs respectively) against SKOV3 cells.

FIG. 8. shows measurements of IFN-gamma secretion by the anti-ROR1 CAR-T cells with humanized scFvs (PMC1182, PMC1183 and PMC1194 expressing PMC857, PMC858, and PMC862 anti-ROR1 CARs respectively) in the presence of SKOV-3 cells or control HL-60 cells.

### DETAILED DESCRIPTION OF THE INVENTION

### Definitions

As used herein, an “antibody” refers to antigen binding proteins of the immune system. A naturally occurring antibody is a glycoprotein comprising at least two heavy (H) chains and two light (L) chains inter-connected by disulfide bonds. Each heavy chain is comprised of a heavy chain variable region (VH) and a heavy chain constant (CH) region. The heavy chain constant region is comprised of three domains, CH1, CH2 and CH3. Each light chain is comprised of a light chain variable region (VL) and a light chain constant CL region. The light chain constant region is comprised of one domain, CL. The VH and VL comprise complementarity determining regions (CDR), interspersed with regions that are more conserved, termed framework regions (FR). Each VH and VL comprises three CDRs and four FRs arranged from amino-terminus to carboxy-terminus in the following order: FR1, CDR1, FR2, CDR2, FR3, CDR3, FR4.

The term “human antibody”, as used herein, is intended to include antibodies having variable regions in which both the framework and CDR regions are derived from human gene sequences. Furthermore, if the antibody contains a constant region, the constant region also is derived from human immunoglobulin sequences.

The term “humanized antibody” refers to antibodies in which CDR sequences derived from the germline of another mammalian species, such as a mouse, have been grafted onto human framework sequences. Additional framework region modifications may be made within the human framework sequences.

As used herein, an “antigen-binding fragment” refers to a protein fragment including Fab fragment, Fab′ fragment, F(ab′)2 fragment, and scFv with antigen-binding activity.

As used herein, a “chimeric antigen receptor (CAR)” is a receptor protein that has been engineered to give T cells a new ability to target a specific protein. The receptor is chimeric because it combines both antigen-binding and T-cell activating functions in a single receptor. CAR is a fusion protein comprising an extracellular antigen-binding domain, a transmembrane domain, and at least one intracellular domain.

As used herein, the “extracellular domain capable of binding to an antigen” means any oligopeptide or polypeptide that can bind to a certain antigen. The “intracellular domain” means any oligopeptide or polypeptide known to function as a domain that transmits a signal to cause activation or inhibition of a biological process in a cell.

As used herein, a “domain” means one region in a polypeptide which is folded into a particular structure independently of other regions.

As used herein, a “single chain variable fragment (scFv)” means a single chain polypeptide derived from an antibody which retains the ability to bind to an antigen. A typical example of an scFv includes an antigen-binding polypeptide which is formed by a recombinant DNA technique and in which Fv regions of immunoglobulin heavy chain (H chain) and light chain (L chain) fragments are linked via a spacer or linker sequence. Various methods for engineering an scFv are known to a person skilled in the art.

As used herein, a “tumor antigen” means a biological molecule having antigenicity, which is a characteristic of a tumor.

The inventors have generated an anti-ROR1 monoclonal antibody that specifically targets the human ROR1 antigen using hybridoma technology. The inventors have produced anti-ROR1 CAR-T cells to target cancer cells overexpressing the ROR1 tumor antigen. The anti-ROR1 CAR-T cells of the present invention have high cytotoxic activity against several cancer cell lines and anti-tumor activity in vivo. Anti-ROR1 CAR-NK cells expressing the same CAR are also contemplated.

In some embodiments, the present invention comprises a monoclonal mouse anti-human ROR1 antibody having the amino acid sequence of SEQ ID NO: 1, or an antigen-binding fragment thereof, comprising a V_{H} having the amino acid sequence of SEQ ID NO: 2 and a V_{L} having the amino acid sequence of SEQ ID NO: 3.

In some embodiments, the present invention comprises a monoclonal mouse anti-human ROR1 antibody or an antigen-binding fragment thereof, comprising a V_{H} having the amino acid sequence of SEQ ID NO: 5 and a V_{L} having the amino acid sequence of SEQ ID NO: 6.

In some embodiments, the present invention comprises a monoclonal humanized anti-human ROR1 antibody or an antigen-binding fragment thereof, comprising a V_{H} having the amino acid sequence of SEQ ID NO: 9 and a V_{L} having the amino acid sequence of SEQ ID NO: 10.

In some embodiments, the present invention comprises a monoclonal humanized anti-human ROR1 antibody or an antigen-binding fragment thereof, comprising a V_{H} having the amino acid sequence of SEQ ID NO: 13 and a V_{L} having the amino acid sequence of SEQ ID NO: 14.

In some embodiments, the present invention comprises a monoclonal humanized anti-human ROR1 antibody or an antigen-binding fragment thereof, comprising a V_{H} having the amino acid sequence of SEQ ID NO: 17 and a V_{L} having the amino acid sequence of SEQ ID NO: 18.

In some embodiments, the monoclonal anti-human ROR1 antibody is generated against the extracellular region of the purified recombinant fragment of human ROR1.

In some embodiments, the invention comprises single-chain variable fragments (scFv) derived from the monoclonal mouse anti-human ROR1 antibody disclosed herein or any of the humanized versions thereof also disclosed herein.

In some embodiments, the invention comprises a chimeric antigen receptor (CAR) fusion protein comprising from N-terminus to C-terminus: (i) a single-chain variable fragment (scFv) against ROR1 disclosed herein, (ii) a transmembrane domain, (iii) at least one co-stimulatory domain, and (iv) an activating domain.

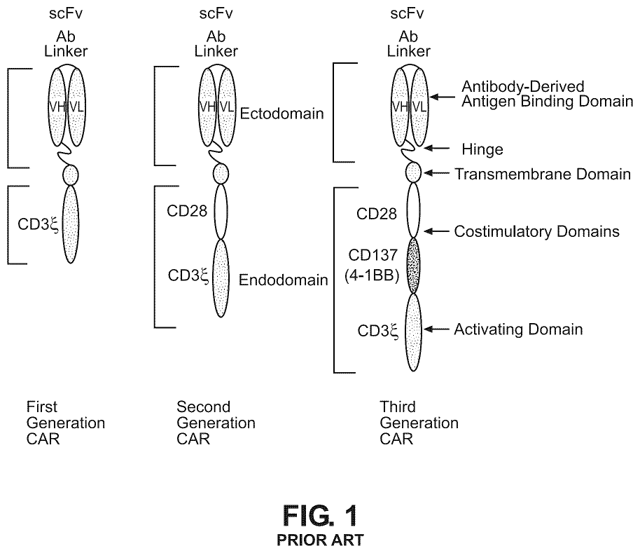

FIG. 1 illustrates the structure of a first-generation CAR lacking the costimulatory domains, the second-generation CAR with one co-stimulatory domain (CD28 or 4-1BB), and the third-generation CAR having two or more co-stimulatory domains (adapted from Goluboskaya et al., (2016) Different Subsets of T Cells, Memory, Effector Functions, and CAR-T Immunotherapy. Cancers (Basel). 2016 Mar. 15; 8 (3). pii: E36).

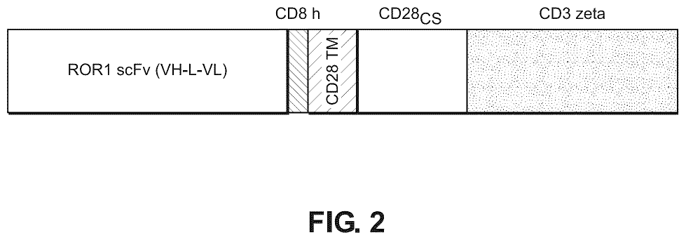

FIG. 2 illustrates the structure of the anti-ROR1 CAR of the present invention. The second-generation CAR was used with either the CD28 or the 4-1BB co-stimulatory domain. (A CAR with the CD28 co-stimulatory domain is shown.) In FIG. 2, “scFv” is a single chain variable fragment; “CD8 h” is a CD8 hinge; “CD28 TM” is a CD28 transmembrane domain; “CD28 cs” is a CD-28 co-stimulatory domain, “CD3-zeta” is a CD3 zeta activation domain, “VH” is a heavy chain variable region, “L” is a linker and “VL” is a light chain variable region. The arrangement of the scFv is shown as V_{H}-linker-V_{L}. In some embodiments, the arrangement is V_{L}-linker-V_{H}.

The co-stimulatory domain can be selected from the group consisting of CD28, 4-1BB (CD137), GITR, ICOS-1, CD27, OX-40 and DAP10 co-stimulatory domains. In some embodiments, the co-stimulatory domain is CD28.

In some embodiments, the activating domain is CD3 zeta (CD3 Z or CD3-zeta, encoded by the CD247 gene.

The transmembrane domain may be derived from a natural polypeptide or may be artificially designed. The transmembrane domain derived from a natural polypeptide can be obtained from any membrane-binding or transmembrane protein. In some embodiments, the transmembrane domain is a transmembrane domain of a protein selected from the group consisting of a T cell receptor α- or β-chain, a CD3-zeta chain, CD28, CD3ε, CD45, CD4, CD5, CD8, CD9, CD16, CD22, CD33, CD37, CD64, CD80, CD86, CD134, CD137, ICOS, CD154, or a GITR. The artificially designed transmembrane domain is a polypeptide mainly comprising hydrophobic residues such as leucine and valine. In some embodiments, a triplet of phenylalanine, tryptophan and valine is found at each end of the synthetic transmembrane domain.

In some embodiments, the CAR comprises a linker between the transmembrane domain and the intracellular domain. In some embodiments, the linker is an oligopeptide or a polypeptide, for example, has a length of 2 to 10 amino acids. A peptide linker generally comprises from about 5 to about 40 amino acids. The linker can be a naturally occurring sequence or an engineered sequence. For example, in some embodiments, the linker is derived from a human protein, e.g., an immunoglobulin selected from IgG, IgA, IgD, IgE, or IgM. In some embodiments, the linker comprises 5-40 amino acids from the CH1, CH2, or CH3 domain of an immunoglobulin heavy chain. In some embodiments, the linker is a glycine and serine rich linker having the sequence (GₓS_{y})ₙ. Additional linker examples and sequences are disclosed in the U.S. Pat. No. 5,525,491 Serine-rich peptide linkers, U.S. Pat. No. 5,482,858 Polypeptide linkers for production of biosynthetic proteins, and a publication WO2014087010 Improved polypeptides directed against IgE.

In some embodiments, the invention comprises one or more nucleic acids encoding the anti-ROR1 CARs. The nucleic acid encoding the CAR can be prepared from an amino-acid sequence of the specified CAR by a conventional method. A nucleotide sequence encoding an amino acid sequence can be obtained using the tools provided to the public by the National Center for Biotechnology Information (NCBI), e.g., from NCBI RefSeq IDs or accession numbers of GenBank for an amino acid sequence of each domain. The nucleic acid of the present invention can be prepared using a standard molecular biological or chemical procedure. In some embodiments, based on the nucleotide sequence, portions of the nucleic acid are synthesized. In some embodiments, the nucleic acid of the present invention is prepared by combining DNA fragments which are obtained from a cDNA library using the polymerase chain reaction (PCR).

In some embodiments, a nucleic acid encoding the CAR of the present invention is inserted into a vector, and the vector is introduced into a cell. In some embodiments, the vector is a viral vector such as a retroviral vector (including an oncoretroviral vector, a lentiviral vector, and a pseudo-type vector), an adenoviral vector, an adeno-associated virus (AAV) vector, a simian virus vector, a vaccinia virus vector, a Sendai virus vector, an Epstein-Barr virus (EBV) vector, or a herpes simplex virus (HSV) vector. In some embodiments, a viral vector lacking the replicating ability so as not to self-replicate in an infected cell is used.

In some embodiments, retroviral particles are prepared using a packaging cell line. In such embodiments, a suitable packaging cell line based on the LTR sequence, and the packaging signal sequence possessed by the viral vector is selected. Examples of the packaging cell lines include PG13 (ATCC CRL-10686), PA317 (ATCC CRL-9078), GP+E-86, GP+envAm-12, and Psi-CRIP. In some embodiments, retroviral particles are prepared using the HEK293 cell line or the HEK293t cell line having high transfection efficiency. One of skill in the art is aware of many kinds of retroviral vectors and packaging cell lines that are commercially available.

A CAR-T cell (or a CAR-NK cell) binds to a specific antigen via the CAR, whereby a signal is transmitted into the cell, and the cell is activated. The activation of the cell expressing the CAR is varied depending on the kind of the cell type and the intracellular domain of the CAR. Activation of the cell can be confirmed based on, for example, release of a cytokine, any improvement of a cell proliferation rate, a change in any cell-surface molecule, or the like. Furthermore, the release of cytotoxic cytokines (IFNγ, TNFα, etc.) from the activated CAR-T cell (or CAR-NK cell) causes destruction of a target cell expressing an antigen which can be detected or measured. In addition, release of a cytokine or change in a cell-surface molecule results in detectable or measurable stimulation of other immune cells, for example, B cells, dendritic cells, NK cells, and macrophages.

In some embodiments, the cell expressing the CAR is used as a therapeutic agent for a disease. The therapeutic agent comprises the cell expressing the CAR as an active ingredient, and it may further comprise a suitable excipient.

In one embodiment, the invention comprises anti-ROR1 scFv-CD28-CD3 zeta-CAR-T (anti-ROR1 CAR-T) cells or anti-ROR1 CAR-NK cells against cancer cells overexpressing ROR1. Anti-ROR1-CAR-T cells or CAR-NK cells express higher cytotoxic activity against ROR1-positive cancer cells compared to non-transduced (no CAR) T cells (or no CAR NK cells) and mock CAR-T cells (or mock CAR-NK cells). The mouse monoclonal anti-human ROR1 antibody disclosed herein detects ROR1 in ROR1-positive cancer cells.

In some embodiments, the invention comprises humanized V_{H} and V_{L} of the mouse monoclonal anti-human ROR1 antibody, a humanized scFv comprising the humanized V_{H} and V_{L}, and CAR-T cells (or CAR-NK cells) harboring a CAR comprising the humanized anti-ROR1 scFv targeting ROR1-positive cells. Without being bound by one particular theory, the inventors perceive at least one advantage of humanizing the mouse anti-ROR1 scFv is potentially reduced immune response to the CAR-T (CAR-NK) cells in humans.

In some embodiments, the anti-ROR1 antibody or antigen binding fragment or derivative thereof (such as an scFv) comprises complementarity determining regions (CDRs). Each of the light chain and the heavy chain of an antibody comprises three CDRs. In some embodiments, CDRs are identified using crystal structure of an antigen-antibody complex. In some embodiments, CDRs are identified using in vitro methods such as phage display. In some embodiments, CDRs are identified using in silico methods, for example, IMGT (Lefranc et al., (2009) IMGT®, The international immunogenetics information system, Nucl. Acids Res. 37:D1006), and Kabat (Kabat et al., (1987) Sequences of Proteins of Immunological Interest, 4th ed., U.S. H.H.S., N.I.H.). In some embodiments, the CDRs are identified using the IMGT tool. In some embodiments, the CDRs are identified using the Kabat tool. In some embodiments, the minimal portions of the CDRs are identified as an overlap of the sequences identified by the IMGT tool and the sequences identified by the Kabat tool.

In some embodiments, the anti-ROR1 scFv comprises the sequence TYA in the CDR1 of the V_{H}. In some embodiments, the anti-ROR1 scFv comprises SEQ ID NO: 41 in the CDR2 of the V_{H}. In some embodiments, the anti-ROR1 scFv comprises SEQ ID NO: 42 in the CDR3 of the V_{H}. In some embodiments, the anti-ROR1 scFv comprises SEQ ID NO: 43 in the CDR1 of the V_{L}. In some embodiments, the anti-ROR1 scFv comprises the sequence RAN in the CDR2 of the V_{L}. In some embodiments, the anti-ROR1 scFv comprises SEQ ID NO: 45 in the CDR3 of the V_{L}.

In some the anti-ROR1 scFv anti-ROR1 comprises the sequence TYA in the CDR1 of the V_{H}, and SEQ ID NO: 41 in the CDR2 of the V_{H}, and SEQ ID NO: 42 in the CDR3 of the V_{H} and further comprises SEQ ID NO: 43 in the CDR1 of the V_{L}, and the sequence RAN in the CDR2 of the V_{L}, and SEQ ID NO: 45 in the CDR3 of the V_{L}.

CDR1 heavy chain
TYA
CDR2 heavy chain
SEQ ID NO: 41
SSGGNT
CDR3 heavy chain
SEQ ID NO: 42
DSYYFGNSVYYAMDY
CDR1 light chain
SEQ ID NO: 43
QDINSY
CDR2 light chain
RAN
CDR3 light chain
SEQ ID NO: 45
LQYDEFPYT

In some embodiments, the anti-ROR1 scFv comprises complementarity determining regions CDR1, CDR2, and CDR3 in the light chain (V_{L}), and CDR1, CDR2, and CDR3 in the heavy chain (V_{H}) and comprises: the sequence TYA in the CDR1 of the V_{H}, SEQ ID NO: 41 in the CDR2 of the V_{H}, SEQ ID NO: 42 in the CDR3 of the V_{H}, SEQ ID NO: 43 in the CDR1 of the V_{L}, the sequence RAN in the CDR2 of the V_{L}, and SEQ ID NO: 45 in the CDR3 of the V_{L}. In some embodiments, in the anti-ROR1 scFv, the CDR1 of the V_{H} consists of the sequence TYA, the CDR2 of the V_{H} consists of SEQ ID NO: 41, the CDR3 of the V_{H} consists of SEQ ID NO: 42, the CDR1 of the V_{L} consists of SEQ ID NO: 43, the CDR2 of the V_{L} consists of the sequence RAN, and the CDR3 of the V_{L} consists of SEQ ID NO: 45.

The humanized anti-ROR1 antibody and the scFv derived therefrom that are disclosed herein can be used for immunotherapy applications: toxin-drug conjugated antibody, monoclonal therapeutic antibody, bispecific antibody, and CAR-T cell (or CAR-NK cell) based immunotherapy.

The anti-ROR1 CAR-T cells (or CAR-NK cells) generated using the anti-ROR1 antibody disclosed herein can be effectively used to target the ROR1 antigen in ROR1-positive cells and tumors. The anti-ROR1CAR-T cells (or CAR-NK cells) can be used clinically against tumor cells, tumors, and cancer stem cells that are resistant to chemotherapy and form aggressive tumors.

The anti-ROR1 CAR-T cells (or CAR-NK cells) can be used in combination with different therapeutic agents: checkpoint inhibitors; targeted therapies, small molecule inhibitors, antibodies and the like. For example, anti-ROR1 CAR-T cells (or CAR-NK cells) can be used in combination with CAR-T (or CAR-NK) cells targeting other tumor antigens or antigens present in the tumor microenvironment (e.g., VEGFR-1-3, PDL-1, CD80). Bi-specific antibodies and scFvs (e.g., bi-specific against ROR1 and CD3), and cells expressing the antibodies and the scFvs can be used to enhance activity of ROR1-targeting therapy.

The anti-ROR1 antibody and its derivatives disclosed herein can be modified with site-directed mutagenesis, e.g., with error-prone PCR for affinity tuning and selected by affinity maturation. Modifications of co-activation domains: CD28, 4-1BB and others can be used to increase the efficacy of the CAR generated from the antibody (and its derivatives) disclosed herein. Tag-conjugated anti-ROR1 scFv can be used for CAR generation. First, second and third generation CAR constructs can be made with the same anti-ROR1 scFv disclosed herein.

The anti-ROR1 CAR disclosed herein can be used for generating CAR-T cells, CAR-NK cells and other types of cells such as iPSCs (induced pluripotency stem cells) from which T cells, NK cells, macrophages and other anti-ROR1 CAR-expressing hematopoietic cells, which can target ROR1-positive cancers. The present invention provides T cells, or NK cells, or macrophages, or hematopoietic cells, modified to express the anti-ROR1 CAR.

The CAR-expressing cells disclosed herein can be autologous cells and allogenic cells.

The following examples further illustrate the present invention. These examples are intended merely to be illustrative of the present invention and are not to be construed as being limiting.

### EXAMPLES

The inventors generated anti-ROR1 CAR constructs and cloned the constructs into lentiviral vectors. The CAR construct contains the anti-ROR1 ScFv-CD28-CD3zeta insert (or similar insert with 41BB co-stimulatory domain instead of CD28 domain). CMV, EF1 or MNDU3 promoter can be used to drive expression of CAR construct. The lentiviruses were generated in HEK293t cells and titer was established by RT-PCR. Then equal dose of lentiviruses was used for transduction of T cells, as described in Examples.

### Example 1. Anti-ROR1 scFv Detected the ROR1 Protein by Western Blotting, and the Anti-ROR1 Antibody Detected ROR1 by FACS Staining

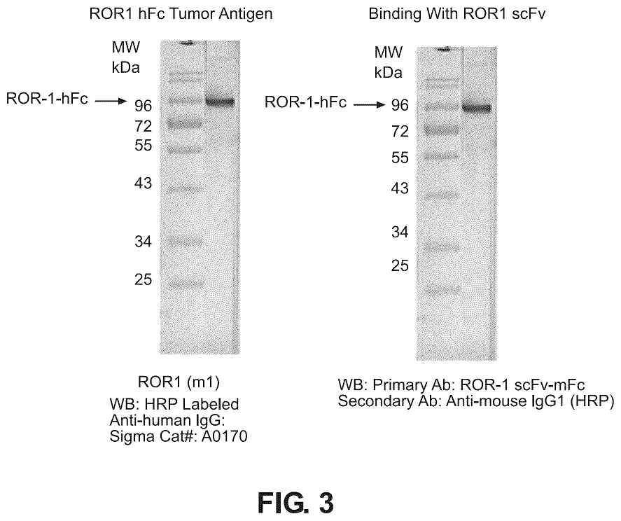

In this example, we generated a mouse monoclonal anti-ROR1 antibody using standard hybridoma technology. The mouse anti-ROR1 antibody (IgG1 type) detected extracellular ROR1 protein by ELISA (data not shown). We sequenced this hybridoma clone 2H6 and generated an scFv using V_{H} and V_{L} (see further in Example 2). We performed a Western blot demonstrating that the anti-ROR1 scFv bound ROR1 extracellular domain fused to human Fc (hFc) protein (FIG. 3, left panel). The ROR1-human Fc fusion protein was detected with an antibody directed against the human Fc domain. FIG. 3, right panel shows ROR1 antigen detected with the anti-ROR1 scFv-mouse Fc fusion as a primary antibody and anti-mouse IgG-HRP as a secondary antibody.

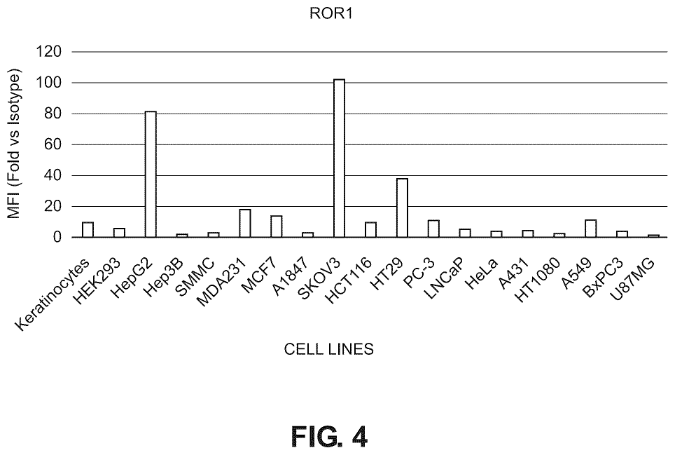

We further performed a fluorescence activated cell sorting (FACS) experiment demonstrating that the mouse anti-ROR1 monoclonal antibody detected elevated expression of ROR1 in several cancer cell lines such as hepatocellular carcinoma (HepG2), breast cancer (MDA231), colon cancer (HT-29), and ovarian cancer (SKOV-3). Normal keratinocytes were used as a negative control (FIG. 4). (MFI: medium fluorescence intensity compared to the isotype).

### Example 2. Sequencing of Anti-ROR1 V_{H}, V_{L}, and CAR Constructs

In this example we sequenced the anti-ROR1 antibody, hybridoma clone 2H6. The sequences of V_{H}, V_{L}, and the scFv are shown below. The structure of the anti-ROR1 scFv is: V_{H}-linker-V_{L} with the linker having the sequence (G₄S)₃ (SEQ ID NO: 46). In the sequences below the sequence starts with the V_{H}; the underline shows the nucleotide sequence of V_{L}; the linker sequence is in italics.

Anti-ROR1 scFv (mouse clone 2H6) nucleotide sequence
(SEQ ID NO: 20):
GTGAAGCTGGTGGAGTCTGGGGGAGGCTTAGTGAAGCCTGGAGGGTCC
CTGAAACTCTCCTGTGCAGCCTCTGGATTCACTTTCAGTACCTATGCCATGTCTTG
GGTTCGCCAGACTCCAGAGAAGAGGCTGGAGTGGGTCGCATCCATTAGTAGTGG
TGGTAACACCTACTATCCAGACAGTGTGAAGGGCCGATTCACCATCTCCAGAGA
TAATGCCAGGCACATCCTGTACCTGCAAATGAGCAGTCTGAGGTCTGAGGACAC
GGCCATGTATTACTGTGCAAGAGATTCTTATTACTTCGGTAATAGCGTTTACTAT
GCTATGGACTACTGGGGTCAAGGAACCTCAGTCACCGTCTCCTCAGGTGGCGGTG
GTTCTGGTGGCGGTGGTTCTGGTGGCGGTGGTTCTGACATCAAGATGACCCAGTCT
CCATCTTCCATGTATGCATCTCTAGGAGAGAGAGTCACTATCACTTGCAAGGCGA
GTCAGGACATTAATAGCTATTTTAGCTGGTTCCAGCAAAAACCAGGGAAATCTCC
TAAGACCCTGATCTATCGTGCAAATAGATTGGTAGATGGGGTCCCATCAAGGTTC
AGTGGCAGTGGATCTGGGCAGGATTATTCTCTCACCATCAGCAGCCTGGAGTATG
AAGATATGGGAATTTATTATTGTCTACAGTATGATGAGTTTCCGTACACGTTCGG
AGGGGGGACCAAACTGGAAATAAAACGG
Anti-ROR1 scFv (mouse clone 2H6) amino acid sequence:
(SEQ ID NO: 1):
VKLVESGGGLVKPGGSLKLSCAASGFTFSTYAMSWVRQTPEKRLEWVASI
SSGGNTYYPDSVKGRFTISRDNARHILYLQMSSLRSEDTAMYYCARDSYYFGNSVY
YAMDYWGQGTSVTVSSGGGGSGGGGSGGGGSDIKMTQSPSSMYASLGERVTITCK
ASQDINSYFSWFQQKPGKSPKTLIYRANRLVDGVPSRESGSGSGQDYSLTISSLEYED
MGIYYCLQYDEFPYTFGGGTKLEIKR
Anti-ROR1 scFv (mouse clone 2H6) V_{H} amino acid sequence
(SEQ ID NO: 2):
VKLVESGGGLVKPGGSLKLSCAASGFTFSTYAMSWVRQTPEKRLEWVASI
SSGGNTYYPDSVKGRFTISRDNARHILYLQMSSLRSEDTAMYYCARDSYYFGNSVY
YAMDYWGQGTSVTVSS
Anti-ROR1 scFv (mouse clone 2H6) V_{L} amino acid sequence
(SEQ ID NO: 3):
DIKMTQSPSSMYASLGER VTITCKASQDINSYFSWFQQKPGKSPKTLIYRA
NRLVDGVPSRFSGSGSGQDYSLTISSLEYEDMGIYYCLQYDEFPYTFGGGTKLEIKR

### Example 3. Anti-ROR1-CAR Sequences with Mouse Anti-ROR1 scFv

In this example we designed a CAR with the scFv derived from the mouse anti-ROR1 antibody 2H6. The scheme of the anti-ROR1 CAR construct is shown in FIG. 2. The lentiviral vector Lenti CMV-MCS-EF1a-puro was used for cloning of the CAR sequence. The CD3 zeta CAR construct was under the control of the CMV promoter. For 4-1BB CAR construct we used another lentiviral vector with MNDU3 promoter to get higher percent of CAR expressing cells.

### A. CD28 as a Co-stimulating Domain

The CAR comprises the following structure: anti-ROR1 ScFv-CD8 hinge-CD28 TM-CD28 co-stimulatory domains CD3 zeta activation domain (FIG. 2). The structure further includes the human CD8 signaling peptide. The anti-ROR1 scFv has the structure V_{H}-Linker (G₄S)₃-V_{L} (“(G₄S)₃” disclosed as SEQ ID NO: 46).

CD8 signaling peptide nucleotide sequence (SEQ ID NO: 21):
ATGGCCTTACCAGTGACCGCCTTGCTCCTGCCGCTGGCCTTGCTGCTCC
ACGCCGCCAGGCCG
CD8 signaling peptide amino-acid sequence (SEQ ID NO: 22):
MALPVTALLLPLALLLHAARP
NheI restriction site: GCTAGC
XhoI restriction site: CTCGAG
CD8 hinge nucleotide sequence (SEQ ID NO: 24):
AAGCCCACCACGACGCCAGCGCCGCGACCACCAACACCGGCGCCCACC
ATCGCGTCGCAGCCCCTGTCCCTGCGCCCAGAGGCGAGCCGGCCAGCGGCGGGG
GGCGCAGTGCACACGAGGGGGCTGGACTTCGCCAGTGAT
CD8 hinge amino acid sequence (SEQ ID NO: 25):
KPTTTPAPRPPTPAPTIASQPLSLRPEASRPAAGGAVHTRGLDFASD
CD28 TM/activation nucleotide sequence (SEQ ID NO: 26):
TTTTGGGTGCTGGTGGTGGTTGGTGGAGTCCTGGCTTGCTATAGCTTGC
TAGTAACAGTGGCCTTTATTATTTTCTGGGTGAGGAGTAAGAGGAGCAGGCTCCT
GCACAGTGACTACATGAACATGACTCCCCGCCGCCCCGGGCCCACCCGCAAGCA
TTACCAGCCCTATGCCCCACCACGCGACTTCGCAGCCTATCGCTCC
CD28 TM/activation amino acid sequence (SEQ ID NO: 27):
FWVLVVVGGVLACYSLLVTVAFIIFWV/RSKRSRLLHSDYMNMTPRRPGP
TRKHYQPYAPPRDFAAYRS
CD3 zeta nucleotide sequence (SEQ ID NO: 28):
AGAGTGAAGTTCAGCAGGAGCGCAGACGCCCCCGCGTACCAGCAGGG
CCAGAACCAGCTCTATAACGAGCTCAATCTAGGACGAAGAGAGGAGTACGATGT
TTTGGACAAGAGACGTGGCCGGGACCCTGAGATGGGGGGAAAGCCGCAGAGAA
GGAAGAACCCTCAGGAAGGCCTGTACAATGAACTGCAGAAAGATAAGATGGCG
GAGGCCTACAGTGAGATTGGGATGAAAGGCGAGCGCCGGAGGGGCAAGGGGCA
CGATGGCCTTTACCAGGGTCTCAGTACAGCCACCAAGGACACCTACGACGCCCT
TCACATGCAGGCCCTGCCCCCTCGC
CD3 zeta amino acid sequence (SEQ ID NO: 29):
RVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATKDTYD
ALHMQALPPR
EcoRI restriction site: GAATTC
Anti-ROR1 CAR (mouse) nucleotide sequence (SEQ ID NO: 30):
ATGGCCTTACCAGTGACCGCCTTGCTCCTGCCGCTGGCCTTGCTGCTCC
ACGCCGCCAGGCCGGCTAGCGTGAAGCTGGTGGAGTCTGGGGGAGGCTTAGTGA
AGCCTGGAGGGTCCCTGAAACTCTCCTGTGCAGCCTCTGGATTCACTTTCAGTAC
CTATGCCATGTCTTGGGTTCGCCAGACTCCAGAGAAGAGGCTGGAGTGGGTCGC
ATCCATTAGTAGTGGTGGTAACACCTACTATCCAGACAGTGTGAAGGGCCGATTC
ACCATCTCCAGAGATAATGCCAGGCACATCCTGTACCTGCAAATGAGCAGTCTG
AGGTCTGAGGACACGGCCATGTATTACTGTGCAAGAGATTCTTATTACTTCGGTA
ATAGCGTTTACTATGCTATGGACTACTGGGGTCAAGGAACCTCAGTCACCGTCTC
CTCAGGTGGCGGTGGTTCTGGTGGCGGTGGTTCTGGTGGCGGTGGTTCTGACATC
AAGATGACCCAGTCTCCATCTTCCATGTATGCATCTCTAGGAGAGAGAGTCACTA
TCACTTGCAAGGCGAGTCAGGACATTAATAGCTATTTTAGCTGGTTCCAGCAAAA
ACCAGGGAAATCTCCTAAGACCCTGATCTATCGTGCAAATAGATTGGTAGATGG
GGTCCCATCAAGGTTCAGTGGCAGTGGATCTGGGCAGGATTATTCTCTCACCATC
AGCAGCCTGGAGTATGAAGATATGGGAATTTATTATTGTCTACAGTATGATGAGT
TTCCGTACACGTTCGGAGGGGGGACCAAACTGGAAATAAAACGGCTCGAGAAGC
CCACCACGACGCCAGCGCCGCGACCACCAACACCGGCGCCCACCATCGCGTCGC
AGCCCCTGTCCCTGCGCCCAGAGGCGAGCCGGCCAGCGGCGGGGGGCGCAGTGC
ACACGAGGGGGCTGGACTTCGCCAGTGATAAGCCCTTTTGGGTGCTGGTGGTGG
TTGGTGGAGTCCTGGCTTGCTATAGCTTGCTAGTAACAGTGGCCTTTATTATTTTC
TGGGTGAGGAGTAAGAGGAGCAGGCTCCTGCACAGTGACTACATGAACATGACT
CCCCGCCGCCCCGGGCCCACCCGCAAGCATTACCAGCCCTATGCCCCACCACGC
GACTTCGCAGCCTATCGCTCCAGAGTGAAGTTCAGCAGGAGCGCAGACGCCCCC
GCGTACCAGCAGGGCCAGAACCAGCTCTATAACGAGCTCAATCTAGGACGAAGA
GAGGAGTACGATGTTTTGGACAAGAGACGTGGCCGGGACCCTGAGATGGGGGG
AAAGCCGCAGAGAAGGAAGAACCCTCAGGAAGGCCTGTACAATGAACTGCAGA
AAGATAAGATGGCGGAGGCCTACAGTGAGATTGGGATGAAAGGCGAGCGCCGG
AGGGGCAAGGGGCACGATGGCCTTTACCAGGGTCTCAGTACAGCCACCAAGGAC
ACCTACGACGCCCTTCACATGCAGGCCCTGCCCCCTCGCTAA
Anti-ROR1 CAR (mouse) amino acid sequence (SEQ ID NO: 4):
MALPVTALLLPLALLLHAARPASVKLVESGGGLVKPGGSLKLSCAASGFT
FSTYAMSWVRQTPEKRLEWVASISSGGNTYYPDSVKGRFTISRDNARHILYLQMSSL
RSEDTAMYYCARDSYYFGNSVYYAMDYWGQGTSVTVSSGGGGSGGGGSGGGGSD
IKMTQSPSSMYASLGERVTITCKASQDINSYFSWFQQKPGKSPKTLIYRANRLVDGVP
SRESGSGSGQDYSLTISSLEYEDMGIYYCLQYDEFPYTFGGGTKLEIKRLEKPTTTPAP
RPPTPAPTIASQPLSLRPEASRPAAGGAVHTRGLDFASDKPFWVLVVVGGVLACYSL
LVTVAFIIFWVRSKRSRLLHSDYMNMTPRRPGPTRKHYQPYAPPRDFAAYRSRVKFS
RSADAPAYQQGQNQLYNELNLGRREEYDVLDKRRGRDPEMGGKPQRRKNPQEGL
YNELQKDKMAEAYSEIGMKGERRRGKGHDGLYQGLSTATKDTYDALHMQALPPR

We also generated a CAR with the 4-1BB co-stimulatory domain in place of the CD28 activation domain. This construct PMC1195 was cloned in a vector with the KanR gene. The ROR1 scFv was inserted between Nhe I and Xho I sites in the sequence (underlined). The CAR expression was under the control of the MNDU3 promoter.

The nucleotide sequence of the codon-optimized CAR (anti-ROR1 scFv-4-1BB-CD3 zeta) is shown below. The scFv is inserted between Nhe I and Xho I sites (underlined). 4-1BB is in italics followed by the CD3-zeta domain.

Anti-ROR1 CAR (4-1BB in place of CD28 activation domain)
nucleotide sequence (SEQ ID NO: 31):
ATGGCCTTACCAGTGACCGCCTTGCTCCTGCCGCTGGCCTTGCTGCTCC
ACGCCGCCAGGCCGGCTAGCGAAGTGAAGCTTGTCGAATCCGGCGGTGGATTGG
TTAAACCAGGCGGAAGTTTGAAACTGAGTTGTGCTGCTTCTGGTTTTACCTTTAG
CACATACGCTATGTCCTGGGTTAGGCAGACGCCGGAGAAACGATTGGAGTGGTA
GCATCTATTTCTTCTGGCGGCAATACTTATTACCCTGACAGCGTGAAAGGCCGGT
TCACTATTTCTCGAGATAATGCGCGGCACATACTCTATCTCCAGATGTCTTCTCTC
CGCTCAGAGGATACAGCGATGTACTATTGTGCAAGGGATAGTTACTATTTCGGA
AACTCTGTGTATTATGCAATGGATTACTGGGGTCAGGGAACTTCAGTCACAGTAA
GCTCAGGTGGGGGAGGAAGCGGCGGTGGCGGCTCAGGGGGAGGTGGATCTGAT
ATTAAAATGACTCAGTCTCCATCAAGCATGTACGCCTCTCTGGGAGAGCGAGTTA
CTATTACCTGTAAAGCATCACAAGATATTAACTCTTATTTTAGTTGGTTTCAACA
AAAGCCTGGAAAATCACCTAAAACTTTGATTTATAGAGCCAATAGGCTTGTGGA
TGGTGTACCTAGTCGGTTTAGCGGCTCAGGGTCAGGCCAAGACTATTCTTTGACC
ATCTCTTCTCTGGAGTATGAGGACATGGGAATCTATTACTGTCTTCAGTACGATG
AGTTCCCCTATACGTTTGGTGGAGGCACTAAATTGGAGATTAAACTCGAGAAGC
CCACCACGACGCCAGCGCCGCGACCACCAACACCGGCGCCCACCATCGCGTCGC
AGCCCCTGTCCCTGCGCCCAGAGGCGAGCCGGCCAGCGGCGGGGGGCGCAGTGC
ACACGAGGGGGCTGGACTTCGCCAGTGATAAGCCCTTTTGGGTGCTGGTGGTGG
TTGGTGGAGTCCTGGCTTGCTATAGCTTGCTAGTAACAGTGGCCTTTATTATTTTC
TGGGTGAAACGGGGCAGAAAGAAACTCCTGTATATATTCAAACAACCATTTATGAGAC
CAGTACAAACTACTCAAGAGGAAGATGGCTGTAGCTGCCGATTTCCAGAAGAAGAAGA
AGGAGGATGTGAACTGAGAGTGAAGTTCAGCAGGAGCGCAGACGCCCCCGCGTA
CCAGCAGGGCCAGAACCAGCTCTATAACGAGCTCAATCTAGGACGAAGAGAGG
AGTACGATGTTTTGGACAAGAGACGTGGCCGGGACCCTGAGATGGGGGGAAAGC
CGCAGAGAAGGAAGAACCCTCAGGAAGGCCTCTACAATGAACTGCAGAAAGAT
AAGATGGCGGAGGCCTACAGTGAGATTGGGATGAAAGGCGAGCGCCGGAGGGG
CAAGGGGCACGATGGCCTTTACCAGGGTCTCAGTACAGCCACCAAGGACACCTA
CGACGCCCTTCACATGCAGGCCCTGCCCCCTCGCTAA

### B. 4-1BB as a Co-Stimulating Domain

We also constructed a CAR with the structure (human CD8 signaling peptide-alternative (see below) anti-ROR1 scFv (V_{H}-Linker (G₄S)₃-V_{L} (“(G₄S)₃” disclosed as SEQ ID NO: 46)), CD8 hinge, CD28 transmembrane domain, 4-1BB co-stimulatory domain, CD3 zeta activation domain). In the alternative scFv, each segment of the sequence is the same as that in Example 3 (A) except V_{H}, V_{L}. V_{H} is represented by SEQ ID NO: 5 (the first amino acid was E, not present in SEQ ID NO: 2). V_{L} is represented by SEQ ID NO: 6 (the terminal R is removed compared to SEQ ID NO: 3.

Alternative anti-ROR1 scFv amino acid sequence
(SEQ ID NO: 23):
EVKLVESGGGLVKPGGSLKLSCAASGFTFSTYAMSWVRQTPEKRLEWVAS
ISSGGNTYYPDSVKGRFTISRDNARHILYLQMSSLRSEDTAMYYCARDSY
YFGNSVYYAMDYWGQGTSVTVSSGGGGSGGGGSGGGGSDIKMTQSPSSMY
ASLGERVTITCKASQDINSYFSWFQQKPGKSPKTLIYRANRLVDGVPSRF
SGSGSGQDYSLTISSLEYEDMGIYYCLQYDEFPYTFGGGTKLEIK
Anti-ROR1 CAR alternative V_{H} amino acid sequence
(SEQ ID NO: 5):
EVKLVESGGGLVKPGGSLKLSCAASGFTESTYAMSWVRQTPEKRLEWVAS
ISSGGNTYYPDSVKGRFTISRDNARHILYLQMSSLRSEDTAMYYCARDSY
YFGNSVYYAMDYWGQGTSVTVSS

Compared with SEQ ID NO: 2, SEQ ID NO: 5 has an extra E on the N-terminal end.

Anti-ROR1 CAR alternative V_{L} acid sequence
(SEQ ID NO: 6):
DIKMTQSPSSMYASLGERVTITCKASQDINSYFSWFQQKPGKSPKTLIYR
ANRLVDGVPSRFSGSGSGQDYSLTISSLEYEDMGIYYCLQYDEFPYTFGG
GTKLEIK

Compared with SEQ ID NO: 3, SEQ ID NO: 6 is lacking the R at the C-terminal end.

4-1BB domain nucleotide sequence: (SEQ ID NO: 32):
AAACGGGGCAGAAAGAAACTCCTGTATATATTCAAACAACCATTTATGAG
ACCAGTACAAACTACTCAAGAGGAAGATGGCTGTAGCTGCCGATTTCCAG
AAGAAGAAGAAGGAGGATGTGAACTG
4-1BB amino acid sequence (SEQ ID NO: 33):
KRGRKKLLYIFKQPFMRPVQTTQEEDGCSCRFPEEEEGGCEL
Anti-ROR1 CAR (alternative scFv and 4-1BB) amino
acid sequence (SEQ ID NO: 7):
(V_{H} is underlined, the linker (G₄S)₃ (SEQ ID
NO: 46) is in italics, V_{L} is underlined; 4-1BB
domain is in underlined italics.)
MALPVTALLLPLALLLHAARPASEVKLVESGGGLVKPGGSLKLSCAASGF
TESTYAMSWVRQTPEKRLEWVASISSGGNTYYPDSVKGRFTISRDNARHI
LYLQMSSLRSEDTAMYYCARDSYYFGNSVYYAMDYWGQGTSVTVSSGGGG
SGGGGSGGGGSDIKMTQSPSSMYASLGERVTITCKASQDINSYFSWFQQK
PGKSPKTLIYRANRLVDGVPSRFSGSGSGQDYSLTISSLEYEDMGIYYCL
QYDEFPYTFGGGTKLEIKLEKPTTTPAPRPPTPAPTIASQPLSLRPEASR
PAAGGAVHTRGLDFASDKPFWVLVVVGGVLACYSLLVTVAFIIFWVKRGR
KKLLYIFKQPFMRPVQTTQEEDGCSCRFPEEEEGGCELRVKFSRSADAPA
YQQGQNQLYNELNLGRREEYDVLDKRRGRDPEMGGKPQRRKNPQEGLYNE
LQKDKMAEAYSEIGMKGERRRGKGHDGLYQGLSTATKDTYDALHMQALPP
R.

### Example 4. ROR1 CAR with Humanized ROR1 scFv

In this example we humanized the mouse anti-ROR1 V_{H} (SEQ ID NO: 5) and mouse V_{L} (SEQ ID NO: 6) and generated several humanized scFv. We generated CARs with the three scFvs having the same structure as Example 3 (B) with the 4-1BB domain and the CD3 zeta domain. We tested several humanized scFv and selected three scFv based on best performance in functional assays shown below. The humanized scFv were inserted between Nhe I and Xho I sites in CAR sequence.

The three CARs with humanized anti-ROR1 scFv: PMC857, PMC858 and PMC862 are shown below.

A. PMC857 scFv and CAR
Humanized anti-ROR1 scFv PMC857 nucleotide sequence: (SEQ ID NO: 34):
GAA GTA CAG CTT GTT GAA TCA GGT GGT GGT CTT ATT CAG CCA GGA
GGC TCC TTG CGA CTG AGC TGT GCC GCT TCT GGG TTC ACC TTT AGC ACT TAC
GCA ATG AGT TGG GTC CGA CAA GCC CCA GGT AAG GGA TTG GAA TGG GTA
AGT TCC ATT TCC AGC GGA GGG AAC ACT TAT TAC GCC GAT TCT GTG AAA
GGA CGC TTT ACT ATA TCC CGA GAC AAT AGT AAA AAC ACA TTG TAT TTG
CAA ATG AAC TCT TTG AGG GCC GAG GAC ACT GCC GTC TAC TAT TGT GCC
CGC GAC AGC TAT TAT TTC GGC AAC TCT GTG TAT TAC GCG ATG GAT TAC
TGG GGT GCC GGC ACA ACT GTC ACC GTT TCA TCT GGC GGA GGA GGC AGT
GGC GGA GGG GGC TCA GGC GGT GGT GGA AGT GAT ATT CAA ATG ACC CAA
TCA CCC TCT TCA TTG TCT GCA AGC GTA GGT GAC CGA GTC ACG ATA ACC
TGC AAA GCC TCT CAA GAT ATT AAT TCA TAC TTT TCT TGG TTT CAA CAA AAA
CCG GGA AAG GCG CCT AAG TCA TTG ATT TAC CGC GCG AAC CGG TTG GTA
TCA GGA GTA CCG TCA AGA TTC TCA GGG AGT GGG TCA GGC ACA GAT TTC
ACA CTC ACT ATT TCT TCC TTG CAA CCT GAA GAC TTC GCA ACC TAT TAT TGC
TTG CAG TAT GAT GAG TTT CCG TAC ACT TTC GGG GGG GGT ACA AGG CTG
GAG ATC AAA
Humanized anti-ROR1 scFv PMC857 amino acid sequence: (SEQ ID NO: 8):
EVQLVESGGGLIQPGGSLRLSCAASGFTESTYAMSWVRQAPGKGLEWVSSISS
GGNTYYADSVKGRFTISRDNSKNTLYLQMNSLRAEDTAVYYCARDSYYFGNSVYY
AMDYWGAGTTVTVSSGGGGSGGGGSGGGGSDIQMTQSPSSLSASVGDRVTITCKAS
QDINSYFSWFQQKPGKAPKSLIYRANRLVSGVPSRFSGSGSGTDFTLTISSLQPEDFAT
YYCLQYDEFPYTFGGGTRLEIK
Humanized anti-ROR1 scFv PMC857 V_{H} amino acid sequence (SEQ ID NO: 9):
EVQLVESGGGLIQPGGSLRLSCAASGFTESTYAMSWVRQAPGKGLEWVSSISS
GGNTYYADSVKGRFTISRDNSKNTLYLQMNSLRAEDTAVYYCARDSYYFGNSVYY
AMDYWGAGTTVTV
Humanized anti-ROR1 scFv (PMC857) V_{L} amino acid sequence (SEQ ID NO: 10):
DIQMTQSPSSLSASVGDRVTITCKASQDINSYFSWFQQKPGKAPKSLIYRANRL
VSGVPSRFSGSGSGTDFTLTISSLQPEDFATYYCLQYDEFPYTFGGGTRLEIK
Humanized anti-ROR1 CAR scFv PMC857 nucleotide sequence: (SEQ ID NO: 35):
ATGGCCTTACCAGTGACCGCCTTGCTCCTGCCGCTGGCCTTGCTGCTCCACG
CCGCCAGGCCGGCTAGC GAA GTA CAG CTT GTT GAA TCA GGT GGT GGT CTT
ATT CAG CCA GGA GGC TCC TTG CGA CTG AGC TGT GCC GCT TCT GGG TTC
ACC TTT AGC ACT TAC GCA ATG AGT TGG GTC CGA CAA GCC CCA GGT AAG
GGA TTG GAA TGG GTA AGT TCC ATT TCC AGC GGA GGG AAC ACT TAT TAC
GCC GAT TCT GTG AAA GGA CGC TTT ACT ATA TCC CGA GAC AAT AGT AAA
AAC ACA TTG TAT TTG CAA ATG AAC TCT TTG AGG GCC GAG GAC ACT GCC
GTC TAC TAT TGT GCC CGC GAC AGC TAT TAT TTC GGC AAC TCT GTG TAT TAC
GCG ATG GAT TAC TGG GGT GCC GGC ACA ACT GTC ACC GTT TCA TCT GGC
GGA GGA GGC AGT GGC GGA GGG GGC TCA GGC GGT GGT GGA AGT GAT ATT
CAA ATG ACC CAA TCA CCC TCT TCA TTG TCT GCA AGC GTA GGT GAC CGA
GTC ACG ATA ACC TGC AAA GCC TCT CAA GAT ATT AAT TCA TAC TTT TCT TGG
TTT CAA CAA AAA CCG GGA AAG GCG CCT AAG TCA TTG ATT TAC CGC GCG
AAC CGG TTG GTA TCA GGA GTA CCG TCA AGA TTC TCA GGG AGT GGG TCA
GGC ACA GAT TTC ACA CTC ACT ATT TCT TCC TTG CAA CCT GAA GAC TTC GCA
ACC TAT TAT TGC TTG CAG TAT GAT GAG TTT CCG
TACACTTTCGGGGGGGGTACAAGGCTGGAGATCAAACTCGAGAAGCCCACCACG
ACGCCAGCGCCGCGACCACCAACACCGGCGCCCACCATCGCGTCGCAGCCCCTG
TCCCTGCGCCCAGAGGCGAGCCGGCCAGCGGCGGGGGGCGCAGTGCACACGAG
GGGGCTGGACTTCGCCAGTGATAAGCCCTTTTGGGTGCTGGTGGTGGTTGGTGGA
GTCCTGGCTTGCTATAGCTTGCTAGTAACAGTGGCCTTTATTATTTTCTGGGTGAA
ACGGGGCAGAAAGAAACTCCTGTATATATTCAAACAACCATTTATGAGACCAGT
ACAAACTACTCAAGAGGAAGATGGCTGTAGCTGCCGATTTCCAGAAGAAGAAGA
AGGAGGATGTGAACTGAGAGTGAAGTTCAGCAGGAGCGCAGACGCCCCCGCGT
ACCAGCAGGGCCAGAACCAGCTCTATAACGAGCTCAATCTAGGACGAAGAGAG
GAGTACGATGTTTTGGACAAGAGACGTGGCCGGGACCCTGAGATGGGGGGAAA
GCCGCAGAGAAGGAAGAACCCTCAGGAAGGCCTCTACAATGAACTGCAGAAAG
ATAAGATGGCGGAGGCCTACAGTGAGATTGGGATGAAAGGCGAGCGCCGGAGG
GGCAAGGGGCACGATGGCCTTTACCAGGGTCTCAGTACAGCCACCAAGGACACC
TACGACGCCCTTCACATGCAGGCCCTGCCCCCTCGCTAA
Humanized anti-ROR1 CAR (scFv PMC857) amino acid sequence: (SEQ ID NO: 11):
MALPVTALLLPLALLLHAARPASEVQLVESGGGLIQPGGSLRLSCAASGFTEST
YAMSWVRQAPGKGLEWVSSISSGGNTYYADSVKGRFTISRDNSKNTLYLQMNSLR
AEDTAVYYCARDSYYFGNSVYYAMDYWGAGTTVTVSSGGGGSGGGGSGGGGSDI
QMTQSPSSLSASVGDRVTITCKASQDINSYFSWFQQKPGKAPKSLIYRANRLVSGVPS
RFSGSGSGTDFTLTISSLQPEDFATYYCLQYDEFPYTFGGGTRLEIKLEKPTTTPAPRP
PTPAPTIASQPLSLRPEASRPAAGGAVHTRGLDFASDKPFWVLVVVGGVLACYSLLV
TVAFIIFWVKRGRKKLLYIFKQPFMRPVQTTQEEDGCSCRFPEEEEGGCELRVKFSRS
ADAPAYQQGQNQLYNELNLGRREEYDVLDKRRGRDPEMGGKPQRRKNPQEGLYN
ELQKDKMAEAYSEIGMKGERRRGKGHDGLYQGLSTATKDTYDALHMQALPPR
B. PMC858 scFv and CAR
Humanized anti-ROR1 scFv PMC858 nucleotide sequence (SEQ ID NO: 36):
CAG GTA CAA TTG GTA GAG TCC GGC GGA GGG GTT GTT CAG CCA GGA
CGG TCC TTG CGG TTG TCT TGT GCT GCG TCA GGA TTC ACA TTC TCA ACG TA
C GCG ATG TCT TGG GTG CGC CAA GCT CCC GGT AAA GGG CTG GAA TGG GT
G GCC TCA ATC TCA TCT GGA GGG AAC ACT TAC TAC CCT GAT AGT GTT AAA
GGT CGC TTT ACT ATC TCA AGG GAC AAT AGC AAG AAT ACC TTG TAT CTG C
AA ATG AAC TCA CTT AGA GCA GAG GAC ACA GCG GTA TAT TAC TGT GCT A
GA GAC TCA TAT TAT TTC GGC AAC TCC GTT TAT TAC GCG ATG GAT TAC TG
G GGC GCA GGG ACT ACG GTA ACT GTA TCT TCT GGT GGT GGA GGG TCT GG
G GGC GGG GGT AGT GGC GGC GGT GGC AGT GAC ATC CAG ATG ACA CAG TC
T CCG TCT TCA TTG AGT GCA AGC GTC GGC GAT CGG GTT ACC ATT ACG TGT
AAG GCA AGT CAG GAC ATC AAC AGT TAT TTT TCA TGG TTT CAA CAA AAG C
CT GGA AAA GCG CCG AAA TCA CTC ATT TAC CGA GCT AAT AGG CTT GTC TC
T GGC GTT CCG TCT CGC TTC AGT GGA AGT GGG AGC GGT ACT GAT TTT ACC
CTC ACC ATA TCA AGC CTT CAA CCG GAG GAT TTT GCC ACG TAC TAT TGT C
TC CAG TAC GAT GAA TTT CCA TAT ACG TTT GGC GGC GGG ACT CGC TTG GA
G ATT AAA
Humanized anti-ROR1 scFv PMC858 amino acid sequence (SEQ ID NO: 12):
QVQLVESGGGVVQPGRSLRLSCAASGFTFSTYAMSWVRQAPGKGLEWVASIS
SGGNTYYPDSVKGRFTISRDNSKNTLYLQMNSLRAEDTAVYYCARDSYYFGNSVYY
AMDYWGAGTTVTVSSGGGGSGGGGSGGGGSDIQMTQSPSSLSASVGDRVTITCKAS
QDINSYFSWFQQKPGKAPKSLIYRANRLVSGVPSRFSGSGSGTDFTLTISSLQPEDFAT
YYCLQYDEFPYTFGGGTRLEIK
Humanized anti-ROR1 scFv PMC858 V_{H} amino acid sequence: (SEQ ID NO: 13):
QVQLVESGGGVVQPGRSLRLSCAASGFTFSTYAMSWVRQAPGKGLEWVASIS
SGGNTYYPDSVKGRFTISRDNSKNTLYLQMNSLRAEDTAVYYCARDSYYFGNSVYY
AMDYWGAGTTVTVSS
Humanized anti-ROR1 scFv PMC858 V_{L} amino acid sequence (SEQ ID NO: 14):
DIQMTQSPSSLSASVGDRVTITCKASQDINSYFSWFQQKPGKAPKSLIYRANRL
VSGVPSRFSGSGSGTDFTLTISSLQPEDFATYYCLQYDEFPYTFGGGTRLEIK
Humanized anti-ROR1 CAR (scFv PMC858) nucleotide sequence (SEQ ID NO: 37):
ATGGCCTTACCAGTGACCGCCTTGCTCCTGCCGCTGGCCTTGCTGCTCCACG
CCGCCAGGCCGGCTAGCCAGGTACAATTGGTAGAGTCCGGCGGAGGGGTTGTTC
AGCCAGGACGGTCCTTGCGGTTGTCTTGTGCTGCGTCAGGATTCACATTCTCAAC
GTACGCGATGTCTTGGGTGCGCCAAGCTCCCGGTAAAGGGCTGGAATGGGTGGC
CTCAATCTCATCTGGAGGGAACACTTACTACCCTGATAGTGTTAAAGGTCGCTTT
ACTATCTCAAGGGACAATAGCAAGAATACCTTGTATCTGCAAATGAACTCACTTA
GAGCAGAGGACACAGCGGTATATTACTGTGCTAGAGACTCATATTATTTCGGCA
ACTCCGTTTATTACGCGATGGATTACTGGGGCGCAGGGACTACGGTAACTGTATC
TTCTGGTGGTGGAGGGTCTGGGGGCGGGGGTAGTGGCGGCGGTGGCAGTGACAT
CCAGATGACACAGTCTCCGTCTTCATTGAGTGCAAGCGTCGGCGATCGGGTTACC
ATTACGTGTAAGGCAAGTCAGGACATCAACAGTTATTTTTCATGGTTTCAACAAA
AGCCTGGAAAAGCGCCGAAATCACTCATTTACCGAGCTAATAGGCTTGTCTCTGG
CGTTCCGTCTCGCTTCAGTGGAAGTGGGAGCGGTACTGATTTTACCCTCACCATA
TCAAGCCTTCAACCGGAGGATTTTGCCACGTACTATTGTCTCCAGTACGATGAAT
TTCCATATACGTTTGGCGGCGGGACTCGCTTGGAGATTAAACTCGAGAAGCCCAC
CACGACGCCAGCGCCGCGACCACCAACACCGGCGCCCACCATCGCGTCGCAGCC
CCTGTCCCTGCGCCCAGAGGCGAGCCGGCCAGCGGCGGGGGGCGCAGTGCACAC
GAGGGGGCTGGACTTCGCCAGTGATAAGCCCTTTTGGGTGCTGGTGGTGGTTGGT
GGAGTCCTGGCTTGCTATAGCTTGCTAGTAACAGTGGCCTTTATTATTTTCTGGGT
GAAACGGGGCAGAAAGAAACTCCTGTATATATTCAAACAACCATTTATGAGACC
AGTACAAACTACTCAAGAGGAAGATGGCTGTAGCTGCCGATTTCCAGAAGAAGA
AGAAGGAGGATGTGAACTGAGAGTGAAGTTCAGCAGGAGCGCAGACGCCCCCG
CGTACCAGCAGGGCCAGAACCAGCTCTATAACGAGCTCAATCTAGGACGAAGAG
AGGAGTACGATGTTTTGGACAAGAGACGTGGCCGGGACCCTGAGATGGGGGGA
AAGCCGCAGAGAAGGAAGAACCCTCAGGAAGGCCTCTACAATGAACTGCAGAA
AGATAAGATGGCGGAGGCCTACAGTGAGATTGGGATGAAAGGCGAGCGCCGGA
GGGGCAAGGGGCACGATGGCCTTTACCAGGGTCTCAGTACAGCCACCAAGGACA
CCTACGACGCCCTTCACATGCAGGCCCTGCCCCCTCGCTAA
Humanized anti-ROR1 CAR (scFv PMC858) amino acid sequence (SEQ ID NO: 15):
MALPVTALLLPLALLLHAARPASQVQLVESGGGVVQPGRSLRLSCAASGFTFS
TYAMSWVRQAPGKGLEWVASISSGGNTYYPDSVKGRFTISRDNSKNTLYLQMNSLR
AEDTAVYYCARDSYYFGNSVYYAMDYWGAGTTVTVSSGGGGSGGGGSGGGGSDI
QMTQSPSSLSASVGDRVTITCKASQDINSYFSWFQQKPGKAPKSLIYRANRLVSGVPS
RFSGSGSGTDFTLTISSLQPEDFATYYCLQYDEFPYTFGGGTRLEIKLEKPTTTPAPRP
PTPAPTIASQPLSLRPEASRPAAGGAVHTRGLDFASDKPFWVLVVVGGVLACYSLLV
TVAFIIFWVKRGRKKLLYIFKQPFMRPVQTTQEEDGCSCRFPEEEEGGCELRVKFSRS
ADAPAYQQGQNQLYNELNLGRREEYDVLDKRRGRDPEMGGKPQRRKNPQEGLYN
ELQKDKMAEAYSEIGMKGERRRGKGHDGLYQGLSTATKDTYDALHMQALPPR
C. PMC862 scFv and CAR
Humanized anti-ROR1 scFv PMC862 nucleotide sequence (SEQ ID NO: 38):
CAG GTA CAA CTG GTG GAA TCC GGC GGG GGA GTA GTA CAG CCC GGA
CGA TCT CTT CGA CTC TCA TGT GCA GCG TCC GGG TTC ACT TTT TCT ACC TAC
GCA ATG TCA TGG GTA CGA CAG GCG CCG GGC AAA GGC CTC GAA TGG GTT
GCA TCC ATT TCA TCA GGA GGT AAT ACA TAT TAT CCT GAT TCA GTC AAG
GGC CGA TTC ACG ATT AGT CGA GAT AAT AGC AAG AAC ACT CTC TAC TTG
CAG ATG AAC TCC CTG CGG GCT GAG GAC ACG GCC GTG TAT TAT TGC GCT
CGC GAT AGT TAT TAC TTC GGC AAT TCC GTA TAT TAT GCG ATG GAC TAT
TGG GGC GCC GGT ACT ACC GTG ACT GTT TCC TCT GGT GGG GGT GGG TCC
GGG GGC GGT GGT TCA GGT GGA GGC GGA TCC GAC ATT CAA ATG ACC CAG
TCT CCC TCA AGT TTG TCT GCA TCT GTT GGC GAT AGA GTT ACA ATA ACA TGC
AAA GCC AGT CAA GAC ATC AAC TCA TAC TTC TCC TGG TAT CAA CAA AAG
CCA GGA AAA GCT CCG AAA CTG TTG ATC TAC CGG GCC AAC CGG CTG GTC
ACT GGC GTG CCA TCC CGG TTC AGT GGC AGC GGA AGC GGA ACA GAT TTC
ACG TTT ACC ATC TCT AGC CTC CAA CCG GAG GAC ATC GCA ACA TAC TAT
TGC CTT CAG TAT GAT GAG TTT CCC TAC ACT TTC GGT GGC GGC ACC CGA CTT
GAG ATC AAA
Humanized anti-ROR1 scFv PMC862 amino acid sequence (SEQ ID NO: 16):
QVQLVESGGGVVQPGRSLRLSCAASGFTESTYAMSWVRQAPGKGLEWVASIS
SGGNTYYPDSVKGRFTISRDNSKNTLYLQMNSLRAEDTAVYYCARDSYYFGNSVYY
AMDYWGAGTTVTVSSGGGGSGGGGSGGGGSDIQMTQSPSSLSASVGDRVTITCKAS
QDINSYFSWYQQKPGKAPKLLIYRANRLVTGVPSRFSGSGSGTDFTFTISSLQPEDIAT
YYCLQYDEFPYTFGGGTRLEIK
Humanized anti-ROR1 scFv PMC862 V_{H} amino acid sequence (SEQ ID NO: 17):
QVQLVESGGGVVQPGRSLRLSCAASGFTFSTYAMSWVRQAPGKGLEWVASIS
SGGNTYYPDSVKGRFTISRDNSKNTLYLQMNSLRAEDTAVYYCARDSYYFGNSVYY
AMDYWGAGTTVTVSS
Humanized anti-ROR1 scFv PMC862 V_{L} amino acid sequence (SEQ ID NO: 18):
DIQMTQSPSSLSASVGDRVTITCKASQDINSYFSWYQQKPGKAPKLLIYRANRL
VTGVPSRFSGSGSGTDFTFTISSLQPEDIATYYCLQYDEFPYTFGGGTRLEIK
Humanized anti-ROR1 CAR (scFv PMC862) nucleotide sequence (SEQ ID NO: 39):
ATGGCCTTACCAGTGACCGCCTTGCTCCTGCCGCTGGCCTTGCTGCTCCACG
CCGCCAGGCCGGCTAGCCAGGTACAACTGGTGGAATCCGGCGGGGGAGTAGTAC
AGCCCGGACGATCTCTTCGACTCTCATGTGCAGCGTCCGGGTTCACTTTTTCTAC
CTACGCAATGTCATGGGTACGACAGGCGCCGGGCAAAGGCCTCGAATGGGTTGC
ATCCATTTCATCAGGAGGTAATACATATTATCCTGATTCAGTCAAGGGCCGATTC
ACGATTAGTCGAGATAATAGCAAGAACACTCTCTACTTGCAGATGAACTCCCTGC
GGGCTGAGGACACGGCCGTGTATTATTGCGCTCGCGATAGTTATTACTTCGGCAA
TTCCGTATATTATGCGATGGACTATTGGGGCGCCGGTACTACCGTGACTGTTTCC
TCTGGTGGGGGGGGTCCGGGGGCGGTGGTTCAGGTGGAGGCGGATCCGACATT
CAAATGACCCAGTCTCCCTCAAGTTTGTCTGCATCTGTTGGCGATAGAGTTACAA
TAACATGCAAAGCCAGTCAAGACATCAACTCATACTTCTCCTGGTATCAACAAA
AGCCAGGAAAAGCTCCGAAACTGTTGATCTACCGGGCCAACCGGCTGGTCACTG
GCGTGCCATCCCGGTTCAGTGGCAGCGGAAGCGGAACAGATTTCACGTTTACCA
TCTCTAGCCTCCAACCGGAGGACATCGCAACATACTATTGCCTTCAGTATGATGA
GTTTCCCTACACTTTCGGTGGCGGCACCCGACTTGAGATCAAACTCGAGAAGCCC
ACCACGACGCCAGCGCCGCGACCACCAACACCGGCGCCCACCATCGCGTCGCAG
CCCCTGTCCCTGCGCCCAGAGGCGAGCCGGCCAGCGGCGGGGGGCGCAGTGCAC
ACGAGGGGGCTGGACTTCGCCAGTGATAAGCCCTTTTGGGTGCTGGTGGTGGTTG
GTGGAGTCCTGGCTTGCTATAGCTTGCTAGTAACAGTGGCCTTTATTATTTTCTGG
GTGAAACGGGGCAGAAAGAAACTCCTGTATATATTCAAACAACCATTTATGAGA
CCAGTACAAACTACTCAAGAGGAAGATGGCTGTAGCTGCCGATTTCCAGAAGAA
GAAGAAGGAGGATGTGAACTGAGAGTGAAGTTCAGCAGGAGCGCAGACGCCCC
CGCGTACCAGCAGGGCCAGAACCAGCTCTATAACGAGCTCAATCTAGGACGAAG
AGAGGAGTACGATGTTTTGGACAAGAGACGTGGCCGGGACCCTGAGATGGGGG
GAAAGCCGCAGAGAAGGAAGAACCCTCAGGAAGGCCTCTACAATGAACTGCAG
AAAGATAAGATGGCGGAGGCCTACAGTGAGATTGGGATGAAAGGCGAGCGCCG
GAGGGGCAAGGGGCACGATGGCCTTTACCAGGGTCTCAGTACAGCCACCAAGGA
CACCTACGACGCCCTTCACATGCAGGCCCTGCCCCCTCGCTAA
Humanized anti-ROR1 CAR (scFv PMC862) amino acid sequence (SEQ ID NO: 19):
MALPVTALLLPLALLLHAARPASQVQLVESGGGVVQPGRSLRLSCAASGFTFS
TYAMSWVRQAPGKGLEWVASISSGGNTYYPDSVKGRFTISRDNSKNTLYLQMNSLR
AEDTAVYYCARDSYYFGNSVYYAMDYWGAGTTVTVSSGGGGSGGGGSGGGGSDI
QMTQSPSSLSASVGDRVTITCKASQDINSYFSWYQQKPGKAPKLLIYRANRLVTGVP
SRFSGSGSGTDFTFTISSLQPEDIATYYCLQYDEFPYTFGGGTRLEIKLEKPTTTPAPRP
PTPAPTIASQPLSLRPEASRPAAGGAVHTRGLDFASDKPFWVLVVVGGVLACYSLLV
TVAFIIFWVKRGRKKLLYIFKQPFMRPVQTTQEEDGCSCRFPEEEEGGCELRVKFSRS
ADAPAYQQGQNQLYNELNLGRREEYDVLDKRRGRDPEMGGKPQRRKNPQEGLYN
ELQKDKMAEAYSEIGMKGERRRGKGHDGLYQGLSTATKDTYDALHMQALPPR

### Example 5. Producing the CAR Using Lentiviral Vectors

In this example, CARs containing the three humanized scFvs of Example 4 were packaged into lentiviral vectors. The lentiviruses were produced by the standard procedure using HEK293 cells as described in Goluboskaya et al., (2016) Different Subsets of T Cells, Memory, Effector Functions, and CAR-T Immunotherapy. Cancers (Basel). 2016 Mar. 15; 8 (3). pii: E36.

### Example 6. Peripheral Blood Mononuclear Cell (PBMC) Isolation from Whole Blood

In this example PBMCs were isolated from whole blood for the purpose of producing CAR-T cells. Whole blood (Stanford Hospital Blood Center, Stanford, Cal.) was collected from individuals or mixed-donor samples (depending on the amount of blood required) in 10 mL heparin vacutainers (Becton Dickinson, San Jose, Cal.). Approximately 10 ml of whole anti-coagulation-treated blood was mixed with sterile phosphate buffered saline (PBS pH 7.4, Ca²⁺ and Mg²⁺-free) for a total volume of 20 ml in a 50 ml conical centrifuge tube. The layer of cells containing PBMCs seen at the diluted plasma/Ficoll interface was removed very carefully, avoiding any Ficoll, washed twice with PBS, and centrifuged at 200×g for 10 min at room temperature. Cells were counted with a hemocytometer. The PBMCs were washed once with CAR-T medium (AIM V-AlbuMAX (BSA) (Life Technologies, San Diego, Cal.) with 5% AB serum and 1.25 ug/mL amphotericin B (Gemini Bioproducts, Woodland, Cal.), 100 U/mL penicillin, and 100 μg/mL streptomycin and used for experiments or frozen at −80° C.

### Example 7. T-Cell Activation from PBMC

Isolated PBMC were washed with once 1×PBS (pH7.4, no Ca²⁺/Mg²⁺), and in CAR-T medium (Example 6), in the absence of human interleukin-2 (huIL2) at a concentration of 5×10⁵ cells/mL, then resuspended to a final concentration of 5×10⁵ cells/mL in CAR-T medium with 300 U/mL huIL2 (from a 1000× stock; Invitrogen, Carlsbad, Cal.). PBMC and beads (for T cell activation) were then mixed at a 1:1 bead-to-cell ratio, by transferring 25 μL of beads to 1 mL of PBMC and incubated at 37° C. in the presence of CO₂ for 24 hr before viral transduction.

### Example 8. T-Cell Transduction and Expansion

Following activation of PBMC, 5×10⁶ lentiviruses were added to 5×10⁵T cells (MOI 10:1), and 2 μL/mL of media of Transplus (Alstem, Richmond, Cal.) to final dilution of 1:500. The cells were incubated for an additional 24 hours before repeating the addition of virus. The cells were then grown in the presence of 300 U/mL of IL-2 for a period of 12-14 days (total incubation time was dependent on the final umber of CAR-T cells required). Cell numbers were analyzed every 2-3 days, with media being added at that time to dilute the cell suspension to 1×10⁶ cells/ml.

### Example 9. Transduction of T Cells and CAR Verification by FACS

The cells from Example 8 were washed and suspended in FACS buffer (PBS plus 0.1% sodium azide and 0.4% BSA). Cells were then divided into 1×10⁶ cell aliquots. Fc receptors were blocked with normal goat IgG (Life Technologies, San Diego, Cal.). Biotin-labeled polyclonal goat anti-mouse F(ab)₂ antibodies were used to detect mouse anti-ROR1 scFv; biotin-labeled normal polyclonal goat IgG antibodies also served as an isotype control. The cells were incubated at 4° C. for 25 minutes and washed once with FACS buffer. After staining the cells with anti-F(ab)₂ antibody, phycoerythrin (PE)-labeled streptavidin (BD Pharmingen, San Diego, Cal.) and allophycocyanin (APC)-labeled CD3 (eBiocience, San Diego, Cal.) were used to stain the cells. For humanized anti-ROR1 scFv we also used anti-human F(ab)₂ antibodies (Life Technologies).

### Example 10. Real-Time Cytotoxicity Assay

The cytotoxicity was performed using xCELLigence real-time cell analysis system (Agilent, San Jose, Cal.), according to the manufacturer's protocol as described in Berahovich et al., (2018) CAR-T cells based on Novel BCMA monoclonal antibody block multiple myeloma Cell growth. Cancers (Basel) (9).

### Example 11. CAR-T Cells with Mouse Anti-ROR1 scFv Expressed High Cytotoxic Activity Against ROR1-Positive Cancer Cells

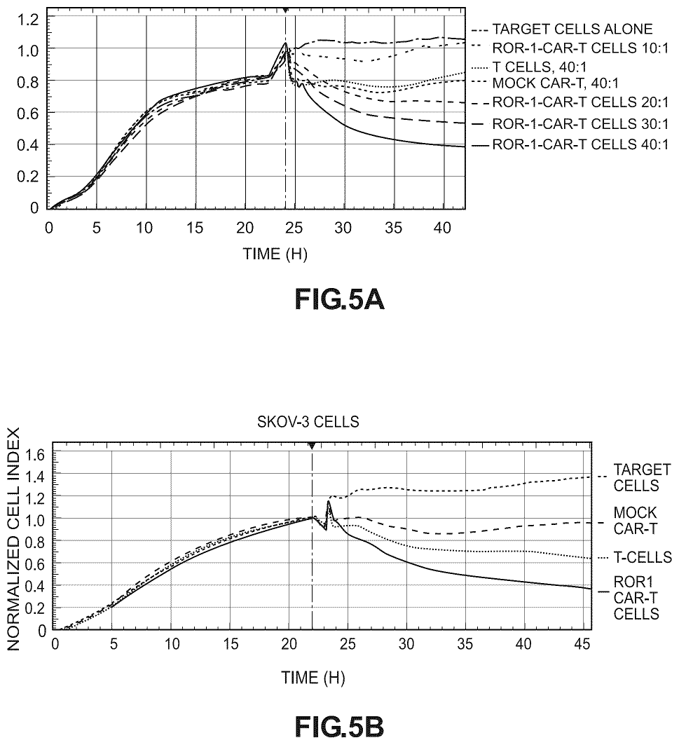

Expression of mouse-scFv anti-ROR1 CAR was confirmed by FACS with anti-mouse Fab antibodies. The CAR-T cells with the CAR containing the mouse anti-ROR1 scfv, CD28 costimulatory domain and CD3 zeta activation domain (see Example 3 (A)) were used in this cytotoxicity assay. The cytotoxicity assay was performed using RTCA impedance-based assay on the xCELLigence system according to manufacturer's conditions. In this assay, the integrity of the target cell monolayer is continually monitored via its impedance in a weak electrical field. Killing of the target cells by the CAR-T cells decreases the monolayer's integrity and, therefore, its impedance. The anti-ROR1 CAR-transduced T cells were added to the target cells at effector: target (E: T) ratios of 10:1, 20:1, 30:1, and 40:1 (FIG. 5, panel A). The CAR-T cells caused a sustained dose-dependent decrease in target cell monolayer impedance. Thus, anti-ROR1-CD28-CD3 CAR-T cells killed the ROR1 positive SKOV-3 ovarian solid tumor cells in a dose-dependent manner.

Similar high cytotoxic activity was observed also with CAR-T cells with the CAR containing the mouse anti-ROR1 scfv, 4-1BB costimulatory domain and CD3 zeta activation (Example 3 (B)) and ROR1-positive SKOV-3 target cells (FIG. 5, panel B).

### Example 12. Anti-ROR1-CAR T Cells (Mouse scFv) Secrete High Level of IFN-Gamma in the Presence of ROR1-Positive Cancer Cells

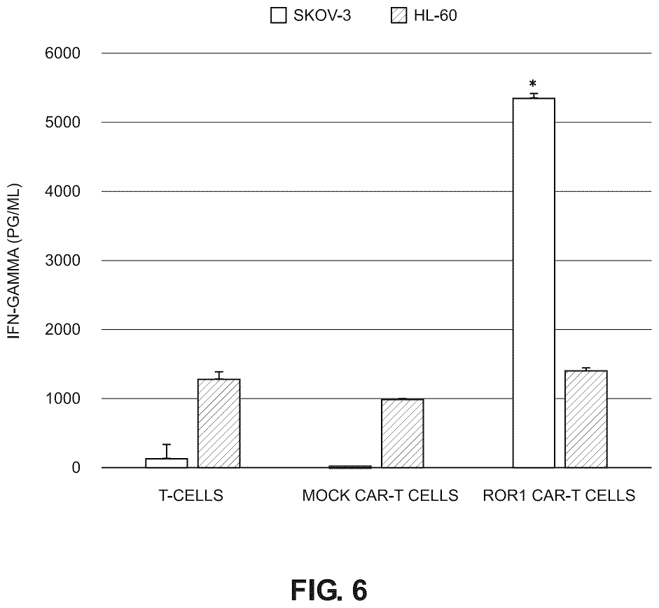

After co-incubation of ROR1-41BB-CD3-CAR-T cells with SKOV-3 cells, we collected the supernatant and performed ELISA with a commercial kit (ThermoFisher Scientific, Waltham, Mass.). As a control we used non-adherent HL-60 ROR1-negative cell line. The anti-ROR1-CAR-T cells secreted significantly higher level of IFN-gamma in the presence of ROR1-positive SKOV-3 cancer cells than in the presence of ROR1-negative control cells, and in comparison to T cells and mock CAR-T cells used as control (P<0.05). (FIG. 6).

### Example 13. Anti-ROR1-CAR T Cells (Humanized scFv) Exhibited Cytotoxicity and Secreted High Level of IFN-Gamma in the Presence of ROR1-Positive Cancer Cells

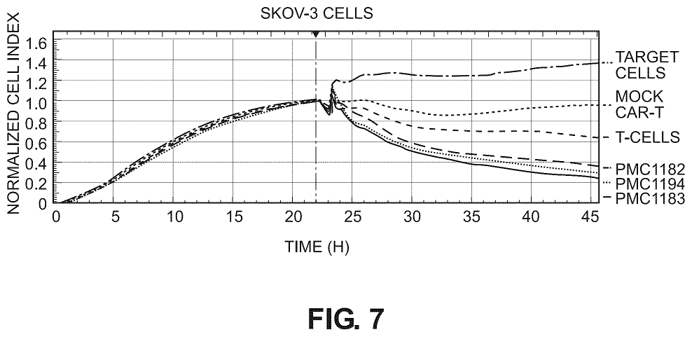

First we tested CAR-T cells with scFvs PMC857, PMC868, or PMC862 (Example 5) in a cytotoxic assay with ROR1-positive cells and showed that the CAR-T cells were highly cytotoxic. Next, we inserted these CAR constructs (PMC857, PMC868, or PMC862) into lentiviral vectors with KanR gene (preferred for clinical usage) instead of AmpR gene. The CAR-T cell clones became clones PMC1182, 1183 and 1194, respectively. The CAR expression in CAR-T cells was about 30% CAR+ as detected by FACS with human Fab. We performed RTCA assay and detected high cytotoxic activity of these CAR-T cells against SKOV-3 (ROR1-positive) cells (FIG. 7).

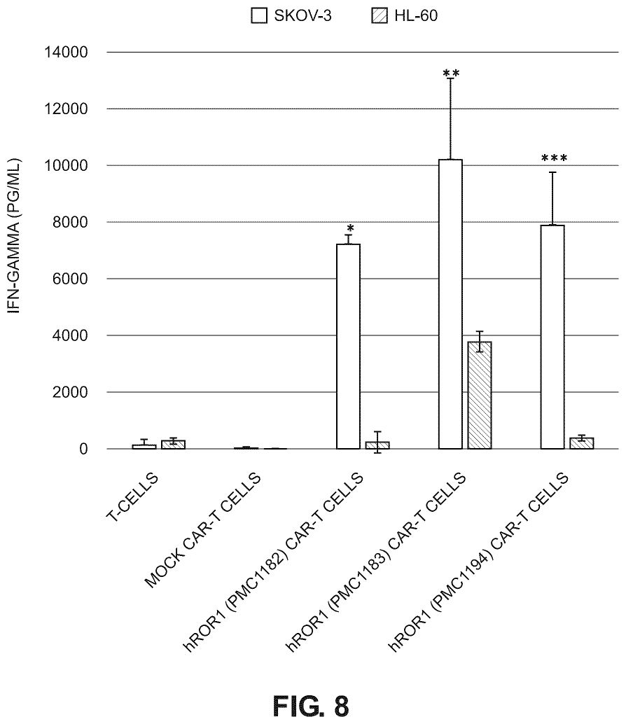

Next we assessed cytokine secretion by the CAR-T cells. After co-incubation of the CAR-T cells with SKOV-3 cells, we collected the culture supernatant and performed ELISA to detect Interferon-Gamma in the supernatant as described in Example 12 using the non-adherent HL-60 ROR1-negative cell line as a control. The anti-ROR1-CAR-T cells secreted significantly higher level of IFN-gamma in the presence of ROR1-positive SKOV-3 cancer cells than in the presence of ROR1-negative control cells, and in comparison to T cells and mock CAR-T cells used as control (P<0.05). (FIG. 8).

While the invention has been described in detail with reference to specific examples, it will be apparent to one skilled in the art that various modifications can be made within the scope of this invention. Thus, the scope of the invention should not be limited by the examples described herein, but by the claims presented below.

1-19. (canceled)

20. A method of making an engineered immune cell expressing an anti-human ROR1 chimeric antigen receptor (CAR), the method comprising expressing in an immune cell a CAR comprising an antibody or antigen-binding fragment comprising a heavy chain V_{H} and a light chain V_{L}, each of the V_{H} and the V_{L} comprising complementarity determining regions (CDRs), wherein a CDR1 of the V_{H} comprises the sequence TYA, a CDR2 of the V_{H} comprises SEQ ID NO: 41, a CDR3 of the V_{H} comprises SEQ ID NO: 42, a CDR1 of the V_{L} comprises SEQ ID NO: 43, a CDR2 of the V_{L} comprises the sequence RAN, and a CDR3 of the V_{L} comprises SEQ ID NO: 45.

21. The method of claim 20, wherein the V_{H} comprises SEQ ID NO: 17, and the V_{L} comprises SEQ ID NO: 18.

22. The method of claim 20, wherein the CAR comprises a single-chain variable fragment (scFv) consisting of the V_{H}, the V_{L}, and a linker.

23. The method of claim 22, wherein the scFv comprises or consists essentially of SEQ ID NO: 16.

24. The method of claim 22, wherein the CAR comprises or consists essentially of the amino acid sequence of SEQ ID NO: 19 without the signaling peptide consisting of SEQ ID NO: 22.

25. The method of claim 20, wherein the cell is isolated from the peripheral blood mononuclear cells (PBMC) fraction of the human whole blood.

26. The method of claim 20, wherein the cell is selected from the group consisting of a T cell, a natural killer (NK) cell and a macrophage.

27. The method of claim 20, wherein the cell is allogeneic.

28. The method of claim 20, wherein the expressing comprises contacting the cell with a construct comprising a vector and a coding sequence for the CAR.

29. The method of claim 28, wherein the coding sequence for the CAR is codon-optimized for expression in human cells.

30. The method of claim 28, wherein the coding sequence for the CAR comprises or consists essentially of SEQ ID NO: 39.

31. The method of claim 28, wherein the vector is a viral vector selected from the group consisting of an AAV vector and a lentiviral vector.

32. The method of claim 20, further comprising assessing the expression of the CAR in the cell.

33. The method of claim 32, wherein the expression of the CAR is assessed by fluorescence-activated cell sorting (FACS).

34. The method of claim 32, wherein the expression of the CAR is assessed by detecting binding of the cells to ROR1-expressing cells.

35. The method of claim 32, wherein the expression of the CAR is assessed by detecting cytokines secreted by the cells in the presence of ROR1-expressing cells.

36. The method of claim 32, wherein the expression of the CAR is assessed by detecting cytotoxic activity of the cells directed against ROR1-expressing cells.
